SEQUENCE LISTING (75 sequences)

SEQ ID NO: 1 - DNA, 11349 bp, Mus musculus
aagaacttaggtgccacgtgccagtcacattgcccttttgctgtttagaatttcctacaaggtatttgtctgctgtttatgaaagaggaggtcaaggctcggggtagaaggccagactccagatgttgccactcccaccgtcagaaccaaattcggtctctccacgtggattgtaactagaaaagacaatggcccctttccaggaaaaaaaaaaaccttctgtcccggggctgccttaaagtgtgtgatttcatgtttagctttcagtgcagaaccatagcagactacattgattatacttttgctgtatgcaaaacatgccactatcttgggggctgcggtgaacccctgccatcaccaggcttaggatctggacgggttgacaaaacatgtttcttattagcgatggttactcccgatgagattaccaggaaagattaattacaaggattcttcttggcatgcccaaactgtttccctcagtcaatatttcaactacttgtcatctattttgtaatacaattttgacttgccattcagattgcaatgacagcaacattttagaaataattggatctctttctaattgagtgggcttgactttcttttcgatgtgtgagtcaggttcgtttcctttgctgtgatgttagcgtggcctttgttcgtgacccagtccttaacagttgagtaacagtccagtcctgctaaggagctgggtcagtctccaaacacctcattgtatccatgtgcaaagttcccagaggcagcttctgtgggtggaactggctaccacgaggcactacttactgcctgctgaaatgaaaacaataaatagcccatacataaagcacttaccgtgtactttgtctaactactttattttattaagtctcaccagggacttactatggtttggataagtatctgtggaggcccatgtgtaaaaggtttggttctcagccaatggctcttttgggaagtactgagccatctcttcggccccaacaatagtgtcttaagcgctattcaaaaccaccgttgtaaaatgtatgaaatgtaactgctgcttcctagatagtttctttacacctacagccatgaggtaggattcaaattgtaccccgtctgtgttagagggttttcttactaatttgtccaatagatgcgagtgagattggcatgtaccacttccaaatggaagtatttgtcatctagggatgggtgctggggccctctctgcctgcccagcaattgcgtgtatagatcttcttagagcagcaccctaacacaaagcagatggggattctgagcctggaaagcagccacccacagcaagctttgcaaatgagaagcaaacttctgctgtttggtagcaattgttacagcagcatagccttgcctaacatgaccataatgtatatcatctatgacctagagccatatgtaaagcttttaataaccatcttgccccattgatacctgaacatcgtttggatagtagcattgtgaaaatggttgctaacatgaagttctgaggggaattgagaaaaaaaatctgtggtcgtttgcatgagaggagccctcatagacttatatggttgaatacttggtctcctgttggttgaactgcttgggaaggattaggaggtgtggccttcttggaggaggtgtgtcactggggataagctttgaggtttcaaaaacctagtaaccacttcccgtgttcctcttggtcaggcatggtatagtacttgggaggcagaagcaagcagatctctctgagttctaggctagccgtctacaccacatagtaagttccaggacaacaagagctacatagtgagactgtctcaaaaaaaagcaacaacaatccagtgtttctctctctgtctcctcagcacatgtgtttgcagctgttcctgcctccgagtctttgctctgccatcatggactccacctctctgaaactgtaagcccaattaggagctttcttttatacgttgccttggtcatggtgttttgtcacaacagaaaagctactaatatggattgttttcataaatctatgtgctaattcacaatctatctctttcgttctataaaaaagaatggtgcaggcagcccagtgatcaatagcaccatccttcaacacaaagaacagcccattatatcttgcaaatagaaaagtaattagctaaactcattctgaaagataggcacaacatgtaaccaccatttctgccaacatgacgattatcaaagggtttacgggcacatcctatgagcaaaggagtatcgctcccagcactgtcaaatatctgacactgtccccttccaaagggatgcttgcaactgcataaactcagcatcattaccctcctgctaagcaaactaggctttccaaggacattctccaggcaccagaaagccctgcttcccagagcacattcctgtctgcacagagggatacttgaaatacatgaatgggaagagtgattcagaaaaataaggactaagccttctcaaattttaaccacaccatttttgaggttaagagaaaagctctattggttttttaaattttaatttaaaaatcaaaaattcctctttttttgaccatgtcagtatggggaatgtcatattgaggaaaacaagaaaaattcttttttgagattcagtgaatctgtccaaatgccagaaaatattatgatcaaatttctacatcaggagaactagagacaatgaggtcctcactcagggacatcccagtcacagtacacagatctcggatattgactagcagcatccttggagacataagcagcagagcagccatcttgagagcagcacacggttgaagcctgtgtagttagagtagatacataatagtaactcattaggacatatgtctccatccttatgacatgtgaaaagttcttacaaattgtaatgtaccatttgcttatttttttatttgtgtgtgtgtgtgtgtgtgtgtgtgtgtgtgtgtgtgtgtgtgctgcctgtgcttgtacgttcatatatggaaagcaaagattgacatatgccttcttttctcaatcactcttcatttttattgtttgagacaggttctctaactgggcctagagctcacttactgaaccagactggctaaccagagaggcccagcgagtctcctgtctcctctcatcacactggggttacacatgggtaccaccacgtccagagctctacagaggctgagaatccacacactggttctcactcttgcctagcagcccttgcttgcgttttctcacattcttttgtcgatgtggtactgaaattaaaagatgtctattgacacctttgaaatggtgccttgcttgcatttttaaacttcatatgtgtattacaaagatttcccacatcatttccttttaagagaagcaatccctgagtgaacaatttagcttgtgtctgcatgtgtcacgtctgtactttttacagtagctacctattggttagggaaatgcaaaaatacattcctacattatctatctcgtgggccatgtgaattccttgtgtatttattataatttagggtgttcgtatacatttttactttccattgaatcagtactctgcacaagtgtgtggagaggtgtgacattctcgggtgtatacacagtaagtgctaactggaccaggcaatgaccacacgcgttcttcagttcttcatggggaaactcaaaacaactcagagcttttaaggcagatgcattcctgtgtgcaagcagtttataaagtgggcaaagaaccatctccctttggcaggttaagctggtcttacatgaagacaagaaactgacctctgaagacagtttgtaggtttctctgactttccataaaacttaacctatcaccactcaaatgggaccaacagttggccggtcttcgctttgtttttgctttttctgggtgacacatattccttcctccccgtggagatcttccctccttatctgtttgtaactaaaaaagttccttgtccgcagaaagctgtaacaccttcaactttaagccaaatgcagaagggaagaagttttacttttatctgccaatactaagctaaaacaaaacaaaacaaaaacaaaaactcttgaaaatgggaacattattaaattaacactacagaccatgatgggagtcttttgattggatagatggaattaaaccccaaggaacctgatcagtgccctttacaaggcacattaggctgcatcttcattgagtatgggagcatcctgtggtgacaatgacatcctatggcaatcactgccatgtggacactataaattccctcaactatcatgggaatgaatatgctacagagtaacaccactctccaaaatgttcaaaaagaaaataatgtgttgatgtgacctgattttgaagctgggtacaaggaaacttatcctttctcccttattttccccttttcacgccccagcccagtaccatctcccctgactaggacaagaacctgcagaaatcagaggttttaacttactaccaccatgcagaaaggactccgacatgtccttggtatctgcttgcaaagaagaatctatcaaaagaaaaccacaggccattctgttcatcttcagctttaacccagaaatatgacagcgggttcctgagcccctgtggataatgcattactacactcaaagcacgattcagctcgggttcttctagggcagaagttgctgagaagcgctttagctaattaaaacttcacagaaacaaggggaaaacaggaaatgttgcacggcacagccaagatgatctctagacacttcacagactggccaaggaccagaaacccaatttaacatttgcaaacgtttctgtgaaatatttgactcctccctgggcaactagagatttacaaactactaagggacattcagcttgctaatgaaaactttataaatacttagtgccagtgaactcagccagggtgctaattctgcctgggctggacaccacagtctgaaaggatacgttgcatgcttagccctagaccagctcaaatcctgcagcttactgaagctgagcctgccataaattgatgactcaatttaataaatgtgacaaatgtctttatttgggctgtggaaaaaatctaccaattataaagacccaaagatgcaaaatatgaattcagggagaccaacagctctccacgctgaggtccatctctgggaggagcacgtgctcacactcgggtctcttctgcccatggggcgctcctatgtgatgcctccttgctattttctgatagccagatagccacagctctcacctgagtgagcacctgtttaacctgtggtccctgctattcacagcagacaatcaagccagcctttctttccaggctctgggctggcaaacaacctcgagccttgggcttttgcttttccggtcggcatttttttaatgtgagtgattgggtgatgcttaaagagtctcatcagcagtcgcaatcccatcagtgagcagtgacctcctgggatcccagagaacagcagggcaagctgggtaggcagcagggcagagacaagggaaggtgccagtaagcctttaattaaggacggaatgcaggctaggtctacactgttgtcctgggtttcaaatttttcaacccccagataaaggaattgtgagtggtggggctaaggtgatggcttaggttataactaaaaggctttgtgttgtgaggaatcgggtgggggggaggatggggggggcatcagtagactcccctgttctagaggcagtgtggccaaagggactcctccctgctggggcttattgtccttggcactctttgtttatttatttattttggtttttagcagaagcaatccagggaaggccaaggcatcctctgctctttcttccaagattcaagagccatttctctccttgaaagaacagagggaatggaaaccagactctccaagataaccagccacaaccagctttcctgatgtgacagacacagagagagagaaagagacagagagacagagggacaggagacgccttcgcaaagcagttttctcatccatcagcagtccgctgctgacaaacctttctgtgaccaaggaagtgcaccgcaccactgaagaaagatcatgggtgtgattgttttggcacgacttagcaactgtgctctattgctatttgtttctgtgtgtgaatcggggtttgtctgtgaagtccagacaaacctagaatgcactacatagccaaagcttgcttgaaactcatgacaatcctcctgcctcagtctcccaatctggaactataggtgagcttcatggggtccaacttctttcagtcatttaaatttttttaatgtgtgattgtgtttgcttgaatgcatgcatgggtgaacgtacgggtggaggacagtgggcacagaaggtactagaagtgcagttacaggggactgtgaaccgcgcactgtggatgctggcaaccgccccagctcctcaacaagagcagcaagtgctcttaaccactgagcggtctctacttcatttcccatttattacagagcttccaaagtatccattcattgacttcttggcctttgtcaacgagcaagttcgatgtacttgggacagagagggggcaggcctccacaggtggacggaatctgccaaacacatggcttactgagatcctggcaattatcgttcttcccagacaggctccttctggtggggaggtagccatcaagtcagacacaggataactcagctggctttgtctctaagggatcatcgcctcacacacccctgactgctgcccaagttccccatggcaaaaactagatgctgggtctaatccccaccctagtagacctccaccaggcagcaaactttcctggcctggtcccttgggttcctggtctggtcccttggctggtttttccaggttggatggctctgaggcccttaacatcttaaagagcaacagctccgttcagttgttagcatagtattcgccctcactctagccaattcttaaagtggaaatacaaagcacctggtctctgtgctcagagttcctcgcacctccaggcgtgggcaccggtcctccctgcgcctgcgcaggaaggtagcttcccctggcatggggacccactagtgacaggacgaagaggagcctaagaaatgaagtcgttgctcctccgagggccaaaggccatcctcttgagcaaccggtaagaatgtaaaagatctcagtttccactggtgagggagagcagctctccttgtactagagacaacctctcgtttgggaaatggcgcccccagatggctgcagcgaatactggagaaagtagtggccctggtaccgggcagccttaccaggcctggaagattcggtacactggagaagacaaccctcacccctggaaagtctgtggataaaagtcaagtaataacacttctggaggcaaccaaaagaacaggacccaaaggcaggcagctagatctctgtgtgacatggttcagttacttaaatcctttgtgcctcaatttccttatgattatggaaaaaaaaaagtgtaacaatcaagggaccagcgaaacagttcagcaggtaagtttgcttgccaccagacctgaccatatgagttttatcttgggggtccatgggtgcaaggagagaaccaaacccctaaagttgtcctctgatctccacaggaatgttgtggccctacaacccccataaataaatgtggagtggggaggcagggggtggttaaggataatgatcacggccacttgctgggtggcagtgaggatttctaaagacaagatgacattggtcgggtggttgctgggtcctcttcctaggggttaggggatgccctactatgtgctgctctagactacaaatcttgacttcaaaacagccttagagcagaaactccccttcctttgacccaggccctgcgtgttaaccaggaaagtggacacctagcacccacattttcccctctaaaggaagtacaggccagtgatgtgaacacttccggggagagcgtgaaaaatgaatctccatttcaagtttgccccctgggcagccaaagctgcatcatcggagaaattaagaaactacacatgcgtgttgtgcttctaggctttcttgctttgagacctttgctgggcttctgtgtgctttcaggacaccattccagagtgcaggaggaagcttgctctcctggcacccaggctgccctgcgacctccttggatgctgggcagaactgggattagcaagctccagacgttggagatccccctaagcatcttagcttgagagcaaagggctcgcctctccagaaatctagcagccagcaaagggttaaccaagcagcagagagactagggcagctttctgtgtccctcaagattcatcaatgccccttaaagacagaaagcaccagatggaaaggcaagaccaccaaaagacagatgtggaggctctttgggcctgcagcatttgagtaggacgttgttggctgggtgctgggatgggggtgggactgagggaagggtccggtactatgagaagcatcctctaagcagaataggagggcctggcactgtgaatcgcactgtttactaactaggaacttcccttcacagggaattgagaggaggctcgacatggactctcagaggcaaaggcttgcgacttttgataggaaacctgtgtacctgggaacctacaggaagcgcagaagagcctaggcgaacccaagcactccaagtgcttgctttgcccgggagttctctcctctgggctacataaacccacacgggaacccggtggggataaccacagagtgtccttgagctgtgcttcacacacagacagtgagtggtctaccctctgaggaaggctactgccctgctccccagcagcgggcttgccgcgcggacacgcctgtcacacaagcatgcaattggctgatgatggacgaaaggagggtgcacgactctagatggagaggaagatggagcttgagcttgagtccaggccgagtggtactgatgttggaacggtctgcgacaacaagctatgtttctgacgacccgggcaagcaagcgggcagaagcgagagcttgagcccggtgttactggagactcccgtaagacttcgggcgacccctcctggcgagcgagacagacgcaggctccccaaacaatgcctaggcccgcggtagtgcgccaggcgcatgggcagcccgctgcactccgaaacacaaaagcgagtgactcaaactagacgaattggtggtgggcttcttgatcttccactcaggggatcggcaggcgggaggtgaccccatggatgtactttggggccacgagtagtgaatacctgagaatactagaattccatttcagcgttcagcctgtgtagctgtttggtttaacttttttgtatttgtgtggtttattttcaaactagtttttacctggggagaaagtctaagcctctggctgcttcagctagacacgggaaggtctagagccaattggatggaaacggctccactcaggcaggccttcagcagagcaggaaagatggagctggacttttaaagactcccattgcgggctccctctgggtgtttacctgcttcgactatggccagaagtgggtaggaaatagcattttatcgtcctctcgtcactccttaaactctcataaactgcgggaccacaaaatgtgctgaccccctaaatcctgagctcaccatccagatggatggaaaatgtaaatcaggcagcaaacatccaacccagctgttaaatccaggaagcagcaacaatgtgtccaccttacacttgaaagaggattgttggggggggggggggcaaagaggggaagtcactacccaggacttgcaaggtcccacttaaagagaggctgactcttaagacaagggaaggtttggggaagagacacccccccccttaacccaccccctgcgcacgtccaaactgtctctctgtactcctttccccgcctggcaccttacaatccacttaccctaacattaggttccctgttctacagagcagaggatggcccgatgggtgaccaggatggcgtgaccaggatgggtgaccaggatggcgtccctggttgttaggggatgaatgggccaactatgctgaggacatgactaactcttgtgaccacagaggtctctgcccgttcctgcccaccttctgaccttggtactcataagccccatttattgctttggtggcctatactgaggtaatcctgaacccctatgtattagagcccgtgcagccttcaagcatagacatctcttcagaggtcaaattttctcctccaaagaggagaaatggcgggcaaattcgtgatgcctctgacaagtcctccacagtgacaaaatcctatagacaccccagcaaaatagcaactccgtgcatggctgcaattccattttataagtcagaatgtgtcgtgaggctgtggaatcacattgctcaagccagtctgctcaacttgggctggagaaccgggaggaaaaagcccaggccacaaagagtctagtactgctggtttatggttactcggggacagggacgcagtgctgagacttaaaggctgctgcacatcgatacgggcgcaggccacgcggggcgtgaaacgcgttccagccctgatccgactgcaaggcagctagcccggctccacgcgtgtcctcgactttcgttgcctcctcctcgctctcccctcgctctccctgggacagggtggcccgcgcttgtctgcttgcaaggcaatcaccagagcgcgcgcgcaggggctgctgcctcggggtgggggcgcagcccgaatttcctgccagcgagcgcgtgaggggcattctcaacgcagccagccccagaaagtagtgaccggcctcctcggattactccggtttggctcctcccttgctccccctcctccacctccagatttgcataaaaaaggccaagaaaactctggctgggccccagcctctgctcactctgctcccccgggtcggagccccccggagctgcgcgcgggcttgcagtgccttgcccgcgccgacctcccggcgcccggcttcgcgcgttcgcgccgcccgctgtccagagcccccacgagcagagcgagggagtcccggacgagctcgagctccggccgcctctcgcttcccccgctcggctccctccgccccccgggggtcgctagtccacgatg

SEQ ID NO: 2 - DNA, 11896 bp, Homo sapiens
ggctaacacaacatttctgctttagtcaataaattctaattcctactctaccaactcattttacaatcagctgctgctgagagaactcattatgtgataagggtgtccctgctcttctgtgccaggttttcctttggtaaaagtgttaataagtagtaaatgccccaaaatgtgaataatattttgatttttaaagtggacacatatacttaatgccactgaactgtacactttaaaatgattaaaatggggccaggcacaatggcttatgcctgtaatcccagcacttcggaaggctgaggcaagcagatcccctgagcccagaagtttgaggctgcagtgagctatgatcatgccactgcactccagcctgggtgacagaacaagaccctgtctctttaaaaaataaaaaatttaaaaaaaatttaaaaaggttaaaatggtaaattttatgttaacatacagtgtattttaccacaataaaaaaaaagtaacacaaagtctgaatcacattaagagagcacctgtaagcatgccagccttctaagagagtaggaaggggcatggttaataaaaccgtgtcggtcagcaaagtagagcaggtaggaatgggaagctgagaattatatcatctgcacccaaagcacagaagcctcttctgataacttatcaagtaaagactgtaaatgcctttatgctaagaatgatactcagtatgaacttatctgtaaaacactagtcatgttgccctcgtggattttacaaggttgcatctttttgctgattaaatcatttctctacaggcactatttatctactgtttctgactgagaaggtcaaggcctggggtggagggccagactccagatgttgtggctcacactgtcagaaccaaatcactctttccacatagatggtctctcaacaacagcacgtgattggaaaagagaatagctccatcccaggaaaagccttctgttctgtgtttgccttaagtgtatgatgttatgtttagccctcagtgcaaagtagtaacaaacttcattgattatacatttgctgtatgcaaagcatactactcagtcttggggctatggcagtacaaaaaaatatactcctaccatcaccaggcttaaagtctggatgggttgacaaaacatgtttctaattagtaatggttagcctgaggagattaccagtgaagattaattacaagggtttttcttggagtgccaaaactgtttccctcagttaatatttcaactacttgtcatctattttgcaatataattttgatttaaaattcaaattccaacaacagtaacaacattttttaaataattagatctctttttaatttagtgagcctgattttcttttgaacatgcaacaaaatgtgagtcagatttgttttccctgcctgggatgatgctgtggagttcattcccagccccatccttaacatccttccctgcctgggatgatactgggagttcattcccagccccatccttaacatccttccctgcctgggatgatactgggagttcattcccagccccatccttaacagcagagcagcactccaactctgctaatgatttgggactttatttatgacctgggtaagtctcctaacaccgcattacatctgcacgataagttcccagaggaagcttctgtgggtggagttggctacactactgtatacctgatgaaataaaaataacacacacagaaaacactatgtacttctcctaagtgctttttcttttttaatcctcgctaggtgctattattaaccccattttatggatgaggaaactaaggcatgaagagtttacatgatttgttcaggatctcacagctggaaagtagtgaaaccagatttgaacacaatttgagttcttcctatcagcctgtcccagatatcagcctaagggacactgatatgtcccagcaccaaatatatatctgaaattaaatcacaggatttgctttctttggttgtaacaatgctgaagaagagaatttaagaaaacatctgcaatcctcatttccccctctctgacgataagactctgactgccctcccctggggagtgttgaagcatgttcttcctctttttgccattccttgttccacagaaaacaagagcaacaaaatatcttggagggttcctttatcttccttccctcaatctatggtatttatctaatctcctggcagtttacatttcatgaacatattttcatagctggatcaccgcgaaatgcaaaaagaaaatttaattcatgaggaccagtaatgcatgctatacaaatatatcataaatgtgtatccctagctacagccccatcacatttcagttggctcaaggattttatttgattaaagaaagttaattcagaaagtacttttctttcttaaaagtgctttactcccccaggttacatttttgtaagcattctctttctcagatctataatataagaagatttttaaagcacattttctgtgctatgtcttcttggtaatgctttgatttcctatagttgttcctatgggaaactcctataggagttaattaaaattatccattagaaatgtaggaaaactttcataggaccatcagcataggcattctagttctagggagttacattatatttctctagctctcctaacagtagcctatttttatttcttcatgtcagttgacattgctagttacaaatgcagtggaactggatcataacctatagattctgctctgatagaaaccaaagcataactatagaaaaagtttcatgtaatatttcccaattgtcagtctgttcactagccccaacttttcagcacgtcaatctataaatctcaacatttattcatcacttgacaggtactgatcaatagtgtcttaagcactattcaaagcaatacatttgaaatgtcaacatatatgtggtaagcatcagtccctaatagccagttcttctccacttctagccactggcaggactacatgttccactcttctgaagctagggctggccttcttgctgatttggctaatagaatatgagtgaaagtgacgtgtcacttctgaaggaaactatttaacttccagtgatggattcaccagccttctctttgcctattcagtgattacaggagcatatcttgatatgggaataccatgatatcaagcagctaagaatgctgaactgggaaaactgctgccctagaaggcccctctcctcaacagaaaactttgcattagtgagagacaaactttagttgtttggtggtgattataatacagtactgtgtgtgatccatgatttgcatcatactgaagcccagagccataggtacaatttctgagtaaactagtcaacttgctctataaatagccaaacatcattttaaccctactgcactaaaaaaaatgagtactaatgtgtgcttctaaggagaattgggaaagatgatttgcataaatcagtatacattttctttatttctataaaagcaagtaatggcatatatcttagtgatcaatgttcacaggaccatccttgtgcataaaggatagccctgtatatcttgtaaatcaaaaatctatttgagctaaattaattctggaaaactatagatagacacaacataaaaatcagtttccagctatttcatgccaacataaagatccttattagaggacttgtaagcacacactctgtgaacaaaaggaacaccacacctctgacagtgtcagaatagatctgacactgtccccttccaaagctacacttaacagtgaacaccttcatcccccagactacgttgcctcaacttccccacaccttccctgtcaatcaccctcatggaaggcagaattgggccttccaagcatgttctccaggaaccagaaaattctgcttcccaaagtacattctctgcacaaagcgatatttgaaatacatggatgggaaaaattgattcagaaaagtaagagctaacccttcccaaattctaaccatgtcatttttgaggttaagagaacagctctttttttccttgcaaatctaaaattcctcttttcaaataagttgatacaggaaatatcatactgaaaagtagaagaaaaacagtttctgagttctggccaatctacccaagtgtcagaggtattataatcaaacttgcatattggcagaaatctggacagtaagatgctcacccagagccatcctgatcacgatgcataattcagccactgtgcaagcagcaccacctgagaacgggaggaaactagagaggagccatcaataacacgcatccactcccgaaactgaatatgacttgaatagtatattcaaaggtaacttattagagcttagatctccattttgacatgtggaaagttgttataaatagtatgtgccatgttactgcatttttatgctgcttcatgtatgtttattttcttaagttaaagaatataacttttttgtgtggatccaagcagtgacattagagatttccacttttctcttgataactttctgtcttgtctgaaatttttacatggagttgtataacttttatacacaaaaagaagcatttggggtgggggaagcaagctgtgagtgaaaagcttcatatgaactcagtttaattttcaattcattcctgttgatttgggaaacacaatatgactatcagcatatttcttagctgttttttggggaaatgcaattgttcaagatccatcattcatctggtaggtcatgagaatttcccaagtatctactacaatttagggtttttaagatggaatgcaattctctgaaagtaaatttataaactgaacagagtataatctccctttgttcatttaagctggtcttatgggagggcaaggaacaaatttctcaagatcttttccagaatttttctctagtagtctgctttctaataaagcttattctatcacaattcaaaaagcaactgaagtcctcagacatgaccagcaaatgaattgttagcctttgcttggttactcctttactgagcaacaccgattatttcactgtatgaggatcttcataagccttcaccctttatccaagtatactgggaataaggctttactctcagaaatatgtaccttttttacgtttaagctaaatgcagacaagcagaaattttacttttacctgcaaatactaagaaaaatccctcaaaggcgtagatcttttttattaccctcatagctcatggtagaggcccaacagtcttttgactggatgaatgaaattaaaccccaaggaacttggggaattccatctttaaggtttcaggttcctaattatatcatctatggttacagagaagagcagagctgaatattgtcctgcctgtggtgatatggtgacatcccatgaaaatcattccagtgactacctatggaatcattcagacaccatgggaatatcctggggaataatgtaccctttcccaaaaaaattcaaaatgcaaggaatctgattttgaagctgggtacaagcaagagtgactccctccttcttcttccctaccccatccctactatccctaattcctctgaatggaacaataactagcataacaagcaaagggtttcaacttattaataccaatggaaaggaatgaaagacaggttttcagtatgtgttccagacaagaattgataacagaaaaacctcagataatcttgctcatcttcaaactctagacaaactgggacataaagaataataacaaatcaactatactcacaaaagcactctcttattttcttccagagcagaacttaatcatgaatttctttaattcattaagatttccaggaaactaagggaaaacagcaaaactctggacaacatagctaagatgatcactagacatttcacagactaccaaggaaccaaatccaccaggaaccaaatttaacatttggaaatattatttctatatagcatgtcactccccattgtacctccagagatttacaaagtattcggaaatattaggcctactaatgaaactttgttggtagtgccaatgaattcagtcagagtgctaaatctgcatatgctggatactatggtttgaaattacagattacacacttagctctaaatcagccaaaaacacacgcttttctgattaacagatttaattgtgttgctgtgaatcctgtgacttactaaagctgagtatatgtcataaaaggataaactgctttaatgattctgacaaatgtctcagtttaagcagcataaaaacctatcgttatagagaaagacccaaacagacacacacacgcagggtatggaaaagttttcctaaaagtcacttcagtaagaatgacaaagggctttttattctgtgatttgtctctggaaagatcatacgtgtgtgcaggaactcagattccatctgttgctttagagcctacaggctttattgtattgactttgtgctcttatctgatggcaataaattctcaccagagtgaaacaccagcttagccagtgtttgaacctgctgttcaaatcagacaatggaacgggtctttttggagacactctgtggcttgtggaccaccctgcgtgaatggccttgggtttttgctgtttccattcaggattttctaatgtgagcaattaagagatacttcatttcaagggtctcatcagcagttgcaacacatcagtgagcaataacctactaggaccccaaaaagagacagggaaagttgggaagaaagggacaggggagagaaaagaagtgccaataaatatttctgtatttaagattgtaacatagatgtacaccggtaacatcaatctcaaattttggaatgcaaagactaaagaatttctactggtccagcaaatttgagaccttatgctataaataaaaggctagagtaaggacgtgtgcactaaacagaaatggaatttcctccccacaagtcgagctcgccgaactgccctcgaggatttttgtgtgtgtggttttttttgtttttttttttttttttggttgttgttttttaatcagaagcagcccagagatacaaaaaagacatgccatgccttcgccccattcattctcccaaaatttctagggctattttcttattgaaggaacaacagaagaaacagaaactggtctctgccaaggcaccaacccacggccagcctccatgatatgcctctatcttcctgagagaagcatagtacaaaatagcgttcagtctattcccagtcagctgtggactaaaacactcatggggtggggaagaacaagaaaatgttctgttgcagaagattatccttgtaggtgtacttgtttttatcattttggagtttggactgttttttagtcattttctactgagcttcaaaacttcgctgtacttggttggggcaaagaggtctcgggaacgtggggttcacactggctttgaacccacagaccagattgcttacacagacctgggcaattacagttctctccaggacagaatccctcagggcgagaaggggtaggccccagactggctcctgctttgcccccaaggaaccgccttctgcgtctccgccgcgcccttcctcctccagcgcgccccttcaggcaagaagggggcgctgagctccgcgttggcctcggcctccctggacccttcgtttgctttccgagcacccatctggggcccagacccttgaaatatttaaggagatacgcgcaagagctcggggtaggagttggcaaactgtttgttctcagactagactcttcctaagatgaggggcaaaggctctccagacgcagactctggtctcgttctcggaaatccgcgtctgaaccgatggggtccgaggagcagaagcgtacacggaggtgcacggccgcgggcacaggtccccccgcgtggcaagaggaagcggcccagccagctgcaggggcgtcctcgttgtggggccgggtagagggagggccaaaggcgaaggaatgaggtcatccccccacccctcgaaaaaaccttcctaaaagtaaaaatgtaagggatctcagcgtaaaggaacctgacttgcaacctcaaagggacaatctctgttttgggaaatggcgcccccagatggctgaatcccatgaaaaaatactcacccggagcagggaaggcctgggtgcacggacccggggagaggtgacgtcacccgcccggctggccagcgcacggctgaaagccaggcggcaacgcggttggagacacccgacaagtcgcgacaggtccccaaggctagcccggtagtcacttcacctcaccgaggcacatgtaacgtcagcagctgtaccacttacggtgtggcccggttcgttacttaacctctccatgcctcagtttcccagtgactgcttgtgtgcaaaatgatcatccgggagccggtgctggattgtgtgaattaaggaactagcattttacacgcatcgttcttctcgctttcactcctcagaaataagggtccatccgctttgattttactacttgagagtgggggtgggaggctcgcttttcccagctcttcgcggaggccaagcggactccctgacaggacagaatctgaacgtgagagtgaaggtcttgcctgtccagaaactcttgtagccagcacaggtttaaacaagaagccaaattgttctggagagattgctgggggctttctttgtgcctcaagcttcttcagtgccctgagcacaggaaacactcaagcagagaagcagagccaaacccaggatacgggaggtcgaggctcttccgtagacctgcagcattggggtgggatgatgttcattctgtgtgtgttctggaccaagcccctctccagggacctatgggcagccccctttaagcaagatgcccggtggagtgggcatccaccatcacttaccctgggcttgggtgaatagattttccgtgccttaaatgggcagggagggggtaaacatggacggtccattggtacaaataaaagcctttggtgggttttgatcaattgcaaggatcgaaggagacctgtggacctgaggtcaactggcagcagagaagagtctgggttcgtgaaggcgccgccgcggtgccgcgccacgtgtgagcatcccagggaccagtctggggaggcgcgctgggatgggcacccagtgtcccatccctgcgctgctcatatgggcagagctccgtgcccgcgtcccagcctccgcgggaggttattgccctgttccccagtgatcaattggaccggtcttagcccaaaagcagaagggagtggggagagcgacttgaggtgggaaaggcagcagcagcgagtgcagggtcgggaccagactaagtggcgtgggttccgggaggagcagcgatgggccgcaggggacgccgcgggccgggcaaacaagctggccagaggtggacactgcgcacggcagggatggagactcaggcgcgactcccagcacctcctggcggcaagccgggcagcgagggctcccaaaacaatgcagccggcgcgcggagcgcgggggccgccagccacttccgggcgtgcggcagcgcggggctcaaaccagccagaccagcttcgggctccttgaactcccaacaaggggctctgcggggaggaggcgaccccagcggagggcgttggggatttaggggttggccggggccggcggggggttgggggggcgtgaaagcgagagggcccttatcccactcccaggcccaagtcgtagcatctctcagagctcacgctctttattagctctttcttttggtgtgcttctgtttcctttgtggtttcattgcaaagtagtttatgttgggaagggggcttttaggcctcttggtgctttcgttagagacgaaaaggccctgaaccaatttggggaaagagaggccttttgcagctaccccgggcccttccccagcagagcacccggaagctgggctcttagggacgcagattcttggagttcagaatgtgcattctctccagatgacgattttcctggcgtgtttacggtcagcagataacactgagtgggcttttgccacccattcattcattacccggctcctctaagcagctggaccgcaaagtgtgcctgataacttgggcctgggagccgccaaggccaacgcatcttccggaaggagggaaattaggaagccggggtctgagcctgtgaatgactcttaagtggtcgggaagtaataacaatgcgcccaccttacatatgaaagaacttcgccctccggggattatctctttggatcttcacagccacccggagacagcggctgaggagagcgaaggcctcctgtccccatagggagactagggacaattaggcaaaaggagcccaccccccaacccagaacttactgtttgcagtcagagacactagatcccaggctccacgagagcagaggccggcctgtttgttcatttctgcacatccagcgcccagcatggtgccccctggtacgtaggggatgaatgagctaattctagccctcgtggatctctaggcaggactccttcctctacctcgctccctcccagcctgcccatcttttgtgcccgtcttttgtgccggccaccagtctttacaaacagtgccctgggggcccatactccagctttccagagcgcacagctgcaagggcggctgtgtgttagagcccgtgatgccttcgagtattgagtcatgccattcatttaagaaggaaaaatctctccaaaacggggaaatggtgggcagtcctgtgtgactggtgagactcttgtaggggcgtttctacaacgacgaaacccttcctaggcactcactccaacagaataacaagcccattttattagtatttcgttttccatgtaaagttctgctcatacgaatatatttataattctgatttttttacggcattggggagcacaccgacaggctgctgaacggtggctggagattcgagggaaaacgaagttcgccgaggcggcctcgggcgggcaggtcccgggctccatcacagggcacacgcggctaccagggacgcagccccccaacacacacacacacacacacacacacacacacacacacacacacaccctctcccactcatgcctggcaacccagcagaaacttcggactggggcaaaacaagcccgggccccggcggcacgcggggctaggcgcgttcccgccagtacctggtcgcgaggccgctcgcggggtgccctgcgtgccccccactcccgcagcccgcgccctgctcgctcactgtgggggcgcagcggccaggcttctctgtttgttgtttaaagaaatcctagggcgggcgagcggcggcatctaggggagggggcgcagccagaattcccttccagcaagcgcgtgaggggcattctcaacgcaaaaccagacccagaaagtagtgaccagccctcctcggattacccttcattggctcctcccttgctccccccaccctccagatttgcataaaaaaggccaagaaaactctggctgtgccccagcaacggctcattctgctcccccgggtcggagccccccggagctgcgcgcgggcttgcagcgcctcgcccgcgctgtcctcccggtgtcccgcttctccgcgccccagccgccggctgccagcttttcggggccccgagtcgcacccagcgaagagagcgggcccgggacaagctcgaactccggccgcctcgcccttccccggctccgctccctctgccccctcggggtcgcgcgcccacgatg

SEQ ID NO: 3 - DNA, 4560 bp, Mus musculus
aaatctttaaattgtattgtgtgagtgcaggtgatttgcttgtgtgtatgtttatacaccacctgtgtgtctggtccacacagaggccagaagagggcatcagatcccccagggctggaggttcagacaatggtgagctgttatgtgggcgcttcaatttaaaccagggtcctctgaaaagcagccagtgctcctaaccattgagccatcactgcagccctgacaatctcttttccctttttagaggaaagatatggctagtcctatgtacagaaacatagggtaaatttaaagtttatttttaaaaatgtttatttattttatgtatacgggtacactgtagctatcttcagacacaccagaagagggcatcagatcccattacagatggttgtgagccaccatgtggttgctgggaattgaaattgaactctggacctctggaagagcagtcagtcagtacttttaactgctgagctgtgtctccagccccctaaaaatttttttcttttaagtagataaaaacactaatttttaaaattgtttagcgttacttaaattattgacaaaacattacattacaacttggaagaggagtgggaagttattgagtcctatgaggtacgggaggcacggatgcgtctcagaggtccaggtgacctcacggttgaaaactcccaaggagtgtgcctagcacagtccttagagattccagaatcagatctccacaaaagttttgttttgtcttaaagtgatgtactggtacatcgataatttgcatgcgacacactgacaatttttttttaaacaccaaatatattgcatgcaactttgacttgtgtccttagttctgcagtatgaattcctatgtccatcaaatatcaaagcaacaacaacaaacccacactcagttctccaggccccccacctgccccttttaaatggcttgaaaatagatataattaagtggtgggctaatgagatggcttagtgggtaatgctgcttgctgcaaagctgggtgaccggattggtagccaggttccatatgtgactgctgacttctgttagttgtcctctggcctccacatacatgctatggcatggttgagcacgcacactaaattaaaagtataaaaatgtttcagaagatgtgagggaatgtctatagtaagcaactgtcttgtttgtagttctgataacagttttaattttaaacgtgtgtgtgtttgtgtgtgtgtgcatgtgcatgtatatgtgtgtgtgcgtgtgcgtgtatatgtgtgtgtgcgtgtgcgtgtgttgggccctcagagtccagaagaataatcagatcccttggagctggagttaaaagttgttgatgtaggtgctaggaaccaaactctggtcctctggaagaacagcaagtgttcttaactgctgagccatttctctggtcctccatgtttatagaaagattactgtcagttaaagtacatttaaataacactatataaaataaaaatgtaagcaaaacagattatgaatgctctgatttctctaaaataccaacctggtttcagttttcttgaaatgttccgaattttgggtttgttgaaggtacttattttagggatacagctcacctgttagtagaaaatgcacatttttttgaacttgtgttttctttgttatcacaaatttttgagaattaagtctcctctcaagtggtttatctccttaagatatttggacttccatttccaccagccagttttcccactgcttcttcaaaaggagcagatagactgtcttcttctgaaagttgtataagccatttaggaaagcagaaagaacaaaaaatataaaagataattaacatactggcagatgccgaagaatattgtgcacacatgacagatgtttatgagaaacaatagagagaaggcacaggagcagacagacaggtgcacacatcctcttctgggaggaatgcgaatccatctctggttttagtaaaaactaatttggattaaaggaaagttgcaagtcccagttgtgatggtgcccacattagatcctagcactagggaggcagaagcaggcagatctctgtgagtttcaggctacctgggtctatatactgagttccaggacagccagggctatatagtgagatgctatttcaaaaacaaaaagaaaaacaaaactaagcaaaacaacagagagggggaagttgggaggttggtggaaggggagagcttgtgagaggatttggtggtaagtgggtatctatctctgaagcaaagttcattcatacctgtggcaatgcgaccatcaaacactctactcctttcttcctaaaaaatgtgagatgaacacaaaatatgtttaacaacctcttgagttcatcgttcaccttcgaattttttacagatccctatttcttttacctaataaagcttctaaatattcttttagtgactgtttataaatctccctttcagtatttgtgtttgtgtgtttgtgtgcatgtgtgtgtgtgtgctttgccatttttcattgaattaatttatataatgtttggctttcttttcttatatgacaccaaataattgtacatcagccagagtttatttattaattaaatggctccctagccaccatctgttgttgatttcctatgaagtagacacacttccttccgaaaacattgaagtcacaaaccgctggtaatgatcattttagagataaaaattccataaactcaagtcagccactgacattccctaagtgccattgagccagcgcaggggattaaagccgtatcctactgtgtgctctgggaagtagagacagactttatctgcctagtattgacagcaggggggtgggcggtgcggaaggctgggtaaatcagggctgctctagtgacgtccggctttgttgtaggttttgcctctgtgctcccgctgtcttctgacactgcaatccctctgctcaatggttttcctctgaaggggctcagtggttgggcctcggcaggacttcccacgtacaatgctcttaaagccagggggcagccggggtggaaacaggagacttctgggaggcgtagcctgggagggatctgttttctattttctcctctgtattgaactcatctggatgggaaccgttctcctgtttactattatcattacctgtgtggggtattttgcttgcacgtttatttcccctgttagaagaaaccgcccgtgggaagagttagataataaatgcaattttcagtaagaacactctgggggtggggggcggaatccaagtgctttaaagtgggctccacgcttcggttttaaaaaagaaaaactcaaaagttcgaattctacagggcaaagaaaaacccggaaggaagcagggagggagggaggaagggaaggaaagaaaaaaaaagaaggagggaagggaggaatcacaccatttccacgtttctgtgggtctatttgttctcttgcgatttctttccctttgtgagggtcaatttctccacggttattccaatcacagaccccccgaagtcattagtcctgccattgctcttaggaggctgctatctctgcgacatgacatttaaagtgactttgctcgcgccttcctgtctgactttctgcaggcggaggtgcgctcgggtttgctgtgaggaaagagctgcgggcaacgagggacggtgtgggctcgcggggcgggatacaggggtgcgcatctctgtggtgcgttgagaccgtttctcccgtggggaccaagggttcgtctatggatccagagccgggggtggagtggggaaaggtgtgcggctcctgtcgggagctgcctggggctacagcatcacagatagacagggtctcacactccagtcccctgaaaactcaaagccttctcggaaggaggagccggagggcaggggaccgcggggcggagctcttgtcggccgaggtgggaaggcgcagctgcgagccaaggcgctgacctcctctgagctcctctggccgctcgcaggatcttcccgaccctgcaggacttggcaaactcccacctccgctcccattagtcctcccacccccaccaaatctcctccctcggaggtcccctatccatctcactttgcagaatttatcgcttcttccaacacctttttgcaacaccccagaactccgagtcccttaactgaatttgacttttgtttttatttctctctggcttcctcttctgccccctcatctgattgatgtgctaaggctgatgtctctgccagagcgagaggaataaatagatgctgcctcgcctagaggcttagacgcttgggaagagcagccggcgcagcgaggcaccgggctccgccaagctagtggaccggacctgggagcacttggatccaagagaactgtgattgtcccaggggtgggggcagctccccaggtcgttgggatcacccctcggaaccgcagggggagacttcggaacgaaagtgtctcccgcgtccgtcgctcggctgcgccctgccccatcctgctgggaccatg

SEQ ID NO: 4 - DNA, 4247 bp, Homo sapiens
ttttgcaaacacattttcttataggaaagatgtgggcagacttattaacacaaacctaggataggtttacagtttatttaaacagatgaaaaaataatgtgaatattcaaattactgacaaaataagacattgcaaaattggggcagggagggggaaattggccaaatttgaattttggtaatatggctatatcaaagtaatatactctagcatgatctttttcttgacattgccacaggaacacctacctgtcaggaagttaaaccaaatgtcaatgatcatggtatcattactcatcctaagaagcttgactggccatcactgggtctgggtgcacttcctaagcagtggttgagcctggaattaacccatagtcccaactaggtgctagggggcgctatgctgcagctctcagacagccaggtgtcctctggctatcataaccaatgcatatcataacctggatatgcattgtgtggagccctttgaagtccttgaatctggtgtccatagattttttgtttccagcagagtattagtatattaataatttggtgggggctcacacctgtaagtgggcagatcatttgagacaaggtgggcagatcatttgagcccaggagtttgagaccagcctgggaaatatggcaaaacttggctgggctcagtggctcacacctgtaattccagcactttgagaggccaaggtggttggatcacctgagtgaggtcaggggttcgagaccagcctggccaacatagtgagccctgtctctactaaaaatacaaaaattagctgggcagggtggtgcgtgcctatagtctcagctactcgggaggctgaggcagcagaatcacttgaacctgggaggtggaggttgcagtgagctgagatcacatcgctgcactccaggctggagacagagcaagattccgtctcagcaaaacaaaaacaatagatgttggtgtggatgtggtgaaagggaatgcttatacactgctggtggaaatgtaaattagtacaacctttatgaaaaacagtgtgaagatttctcaaagaacgaaactggacctacagtttgatacagcaatcccattactgggtatctacccaagtgaaaagacatcaatttgtcagaaggacaactgcatgcatatgtttattgcaacacaattcacaattgcaaagatatagaaccaaactaagtgcccatcaaccaaaaatatggaactaaactaagtgtccatgtccatcaaatgatgagtggataaagaaaatgtggtatatatacaccacggaatactacccagccagaagaaagaatgaaataatgtattttgcagcagtttgggtggagctggaggccattatactaggtgatttaactcaggaatggaaaatgaaatactgtatgttctcacttataaatgagagcaaagctatgggtacaagaaggcatatctttgagaggtataacagactttggagactcacaagtggggagcttgtgagtctactagggaggctgaggcaggagaatctcttgaacccgggaggtggaggttgcagtgagccaagatagtgccactgcactccagcctgggcaacaagagcaaaactccatctcaaaataaataaataaataaataaaggaataatttgtaaaaacaaacactgttcaagggtaactaaccagaaaaatgaacacaactagacaaggtactcaaaagggcttcctcaagacagtgacatttattttgggcttttataaaaacaaatttggattaaagttgtaaggggacctattagacaaagaagatagctgacttttgtgcaggggaaggaaaacagactttgtttcaaatagttgtagaacctttaaaactattggcacatttcttctcacactgtaagacttaagatgaacagaaaaatgtatttagcaaacaatctactacatctactcctgatgttttttcttctaattattgaaaatctcagctatatttttcttatacaaaatagtcccaaaatgttctaatgttgactgtcttttaaagttgtttttcctctaaattttacttgataaatcattttaaattcgtttgtacaatttttgaatttgttctcttacatgacactagataattgtagaccaggcaaagtttgtatactaactgactccctgtctaccatctggtgttccccgcccccgccaccataagtggaacaaacttccttccaaaaaaatttacagcagtcacaaactgctgtttatgatcattttagatataaaattccataactcaagttagcattgacattttataagtgctgtcaagtcactctagggaatgaaaatgagcatctcactatgtgctctgaagtggggacatatttctctgcttgttatggacggtgagagatggctaaattgaagcagagaaatatttcagtgatgcagagcaaaggtgacttcctgttttgtttcaggttttgtttccttctcctctcaatgaaggctgtgttttctgacattcggcaatccttttgctcagtgatttcccattgagagggcatagtgtgaaggcctctgcaggtcattccatttacaatataagctgaaagggtagagtggggcgggggtggggggaagagggagctcattagtggaagaagcagaccaggaggggatctgttttccattttctcctccagattgaactcatctggatggaaaggttttgattgttgttataatcatcattattacctgcgtgatgtcgttttcctacactttttttttaagtgtcgggcagaattgttgctgagaataacaaataatatctgccatgttatccgtgagcaaaatcagaaaaaaaaagtccaagtgtattagagctgttagtttccacgttaacccttaaggagcaaagctcaagagttctaattccactaggtggggggggcgggaatagaaggaaaaaaccccttttccttgcttctggtggcctatttgtagtcatgaacagcatttctttgtttctctctctctttttttttttttttaaaggcaatcctccccccacctcctcccccgcagttattgaaaatggagacctctgtagtcactagctctgggttgatatggctccaccgttgctcgcaggggtctgtgttttccgctacttggacaaagtgacattgcttaagcctttccccccaccaggtctgactttctgcagagccagtgattgcagaggaaaagctgtagtttgcttaaaggaaatacctccaggaaggagggtctcgggtgggttcccaagtggggaactagggggacttttccgtagggaattggggtgggctcttcagtgaggggctaggggctcgtttctggggccaaagacgggttccctagtgtgagggcgcgctcgactcggcgctgtcttggggtctcgcactcgcacggcttcgcaccccaccgcctgcgactcccaggccttctcttccccgggcgcccactccattctcgggaagagcagccggcactggagggcagagactgccccaggggcggagctccctctcaggcgggaggtaggaaagtgcagagccgcccgggcagaggcacagacgtccctgcggggctcctcctgagcgtccctcctgccagccagggtcgcagccgcagcggcggccgcagctcttagcccacacaggacttgtaaactcttactgcacccttctctcccattaggagcttttcctccctccttccccccaacccctctgtcctcctcactttggggaatttaatgctttctttagcatctttttgtgtgcgtgatctaggggaggagacaccccagagctccaactagctctcagctgaattctacttttgtttttatgtcttcctcgcctcctctcgtgtccctcttatctgactgatctgcgaagtctgatgcttctgccagagggagaggaataaatagatgttgctgcttccgaaggcttagacgttgggaaagagcagcctgggcggcaggggcggtggctggagctcggtaaagctcgtgggaccccattgggggaatttgatccaaggaagcggtgattgccgggggaggagaagctcccagatccttgtgtccacttgcagcgggggaggcggagacggcggagcgggccttttggcgtccactgcgcggctgcaccctgccccatcctgccgggatcatg

SEQ ID NO: 5 - DNA, 15 bp, Homo sapiens
gagggccagactcca

SEQ ID NO: 6 - DNA, 15 bp, Homo sapiens
agttggctacactac

SEQ ID NO: 7 - DNA, 15 bp, Homo sapiens
ctctgactgccctcc

SEQ ID NO: 8 - DNA, 15 bp, Homo sapiens
agttggctcaaggat

SEQ ID NO: 9 - DNA, 15 bp, Homo sapiens
gtaaactagtcaact

SEQ ID NO: 10 - DNA, 15 bp, Homo sapiens
cgttgcctcaacttc

SEQ ID NO: 11 - DNA, 15 bp, Homo sapiens
atcattcagacacca

SEQ ID NO: 12 - DNA, 15 bp, Homo sapiens
gcggcccagccagct

SEQ ID NO: 13 - DNA, 15 bp, Homo sapiens
agatggctgaatccc

SEQ ID NO: 14 - DNA, 15 bp, Homo sapiens
cggttggagacaccc

SEQ ID NO: 15 - DNA, 15 bp, Homo sapiens
gcccggtagtcactt

SEQ ID NO: 16 - DNA, 15 bp, Homo sapiens
agcggactccctgac

SEQ ID NO: 17 - DNA, 15 bp, Homo sapiens
tatgggcagccccct

SEQ ID NO: 18 - DNA, 15 bp, Homo sapiens
gtgggaaaggcagca

SEQ ID NO: 19 - DNA, 15 bp, Homo sapiens
gcgggccgggcaaac

SEQ ID NO: 20 - DNA, 15 bp, Homo sapiens
cgggggccgccagcc

SEQ ID NO: 21 - DNA, 15 bp, Homo sapiens
ggccgccagccactt

SEQ ID NO: 22 - DNA, 15 bp, Homo sapiens
agcggccaggcttct

SEQ ID NO: 23 - DNA, 15 bp, Homo sapiens
ggggcgcagccagaa

SEQ ID NO: 24 - DNA, 15 bp, Homo sapiens
ctctggctgtgcccc

SEQ ID NO: 25 - DNA, 15 bp, Homo sapiens
cgccggctgccagct

SEQ ID NO: 26 - DNA, 15 bp, Mus musculus
acgtgccagtcacat

SEQ ID NO: 27 - DNA, 15 bp, Mus musculus
gaaggccagactcca

SEQ ID NO: 28 - DNA, 15 bp, Mus musculus
ctctgcctgcccagc

SEQ ID NO: 29 - DNA, 15 bp, Mus musculus
ggaaagcagccaccc

SEQ ID NO: 30 - DNA, 15 bp, Mus musculus
agccgtctacaccac

SEQ ID NO: 31 - DNA, 15 bp, Mus musculus
atggtgcaggcagcc

SEQ ID NO: 32 - DNA, 15 bp, Mus musculus
gcagagcagccatct

SEQ ID NO: 33 - DNA, 15 bp, Mus musculus
atatgtctccatcct

SEQ ID NO: 34 - DNA, 15 bp, Mus musculus
agttggccggtcttc

SEQ ID NO: 35 - DNA, 15 bp, Mus musculus
gctgggtaggcagca

SEQ ID NO: 36 - DNA, 15 bp, Mus musculus
gaagtccagacaaac

SEQ ID NO: 37 - DNA, 15 bp, Mus musculus
acatggcttactgag

SEQ ID NO: 38 - DNA, 15 bp, Mus musculus
gggaggtagccatca

SEQ ID NO: 39 - DNA, 15 bp, Mus musculus
agctggctttgtctc

SEQ ID NO: 40 - DNA, 15 bp, Mus musculus
gtactagagacaacc

SEQ ID NO: 41 - DNA, 15 bp, Mus musculus
agatggctgcagcga

SEQ ID NO: 42 - DNA, 15 bp, Mus musculus
accgggcagccttac

SEQ ID NO: 43 - DNA, 15 bp, Mus musculus
ctggagaagacaacc

SEQ ID NO: 44 - DNA, 15 bp, Mus musculus
caaaggcaggcagct

SEQ ID NO: 45 - DNA, 15 bp, Mus musculus
cctgggcagccaaag

SEQ ID NO: 46 - DNA, 15 bp, Mus musculus
gcgagcgagacagac

SEQ ID NO: 47 - DNA, 15 bp, Mus musculus
catgggcagcccgct

SEQ ID NO: 48 - DNA, 15 bp, Mus musculus
gaggggaagtcacta

SEQ ID NO: 49 - DNA, 15 bp, Mus musculus
ggggaagagacaccc

SEQ ID NO: 50 - DNA, 15 bp, Mus musculus
gcatggctgcaattc

SEQ ID NO: 51 - DNA, 15 bp, Mus musculus
gactgcaaggcagct

SEQ ID NO: 52 - DNA, 15 bp, Mus musculus
cgttgcctcctcctc

SEQ ID NO: 53 - DNA, 15 bp, Mus musculus
tcaacgcagccagcc

SEQ ID NO: 54 - DNA, 15 bp, Mus musculus
ctctggctgggcccc

SEQ ID NO: 55 - DNA, 15 bp, Homo sapiens
gcttgactggccatc

SEQ ID NO: 56 - DNA, 15 bp, Homo sapiens
ctcagacagccaggt

SEQ ID NO: 57 - DNA, 15 bp, Homo sapiens
gtcagaaggacaact

SEQ ID NO: 58 - DNA, 15 bp, Homo sapiens
aactgactccctgtc

SEQ ID NO: 59 - DNA, 15 bp, Homo sapiens
agatggctaaattga

SEQ ID NO: 60 - DNA, 15 bp, Homo sapiens
aggtgacttcctgtt

SEQ ID NO: 61 - DNA, 15 bp, Homo sapiens
atatggctccaccgt

SEQ ID NO: 62 - DNA, 15 bp, Homo sapiens
ggggaggagacaccc

SEQ ID NO: 63 - DNA, 15 bp, Homo sapiens
ttatgtcttcctcgc

SEQ ID NO: 64 - DNA, 15 bp, Homo sapiens
atctgactgatctgc

SEQ ID NO: 65 - DNA, 15 bp, Homo sapiens
agcagcctgggcggc

SEQ ID NO: 66 - DNA, 15 bp, Homo sapiens
cggtggctggagctc

SEQ ID NO: 67 - DNA, 15 bp, Mus musculus
gaggttcagacaatg

SEQ ID NO: 68 - DNA, 15 bp, Mus musculus
gaagagcagtcagtc

SEQ ID NO: 69 - DNA, 15 bp, Mus musculus
caggagcagacagac

SEQ ID NO: 70 - DNA, 15 bp, Mus musculus
agcagacagacaggt

SEQ ID NO: 71 - DNA, 15 bp, Mus musculus
aaatggctccctagc

SEQ ID NO: 72 - DNA, 15 bp, Mus musculus
gccattgagccagcg

SEQ ID NO: 73 - DNA, 15 bp, Mus musculus
gaagtagagacagac

SEQ ID NO: 74 - DNA, 15 bp, Mus musculus
tcctgtctgactttc

SEQ ID NO: 75 - DNA, 15 bp, Mus musculus
ggggtgggggcagct

(19) United States
(12) Patent Application Publication
(10) Pub. No.: US 2006/0166251 A1
(43) Pub. Date: Jul. 27, 2006

(54) Use of sFRPs as markers of BMP activity

(76) Inventors: Joanne M. Archambault, Medford, MA (US); Scott Jelinsky, Waltham, MA (US); Michael J. Agostino, Andover, MA (US); Li Li, Watertown, MA (US); Howard Seeherman, Cambridge, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/339,549

(22) Filed: Jan. 26, 2006

Related U.S. Application Data
(60) Provisional application No. 60/646,610, filed on Jan. 26, 2005.

(51) Int. Cl. C12Q 1/68

(52) U.S. Cl. 435/6

(57) ABSTRACT

The disclosure provides assay systems for evaluating the presence of bone morphogenetic protein (BMP) activity in a cell by evaluating the gene expression of secreted Frizzled Related Protein 2 and 3 (sFRP2 and sFRP3). The sFRP2 and sFRP3 gene expression may be detected at the RNA or protein levels. The methods include methods for evaluating exogenous and endogenous BMP expression and utilize both genomic sFRP2 and sFRP3 genes and reporter constructs.

### PRIORITY CLAIM

This application claims priority to U.S. Ser. No. 60/646,610, filed Jan. 26, 2005.

### FIELD OF THE INVENTION

This invention concerns methods of evaluating the biological activity of bone morphogenetic proteins (BMPs) by determining whether the BMPs induce the expression of selected secreted Frizzled Related Proteins (sFRPs).

### BACKGROUND OF THE INVENTION

Bone morphogenetic proteins (BMPs) are members of the TGF-β superfamily of growth and differentiation factors. Rosen et al., “Bone Morphogenetic Proteins” Principles of Bone Biology 2:919-928(2002). The first BMPs (BMPs-1-4) were identified by their ability to induce new bone formation in muscle tissue (Urist et al., “Bone Formation By Autoinduction” Science 150:893-99 (1965)). Additional BMPs were cloned by homology screening with the sequences of known BMPs, and have been shown to possess a wide range of growth and differentiation activities, including induction of the growth and differentiation of bone, connective, kidney, heart, and neuronal tissues. Rengachary, “Bone Morphogenetic Proteins: Basic Concepts” Neurosug Focus 13(6):1-6 (2002). See, for example, descriptions of BMPs in the following publications: BMP-2, BMP-3, BMP-4, BMP-5, BMP-6, and BMP-7 (disclosed, for example, in U.S. Pat. Nos. 5,108,922; 5,013,649; 5,116,738; 5,106,748; 5,187,076; and 5,141,905), BMP-8 (disclosed in PCT WO 91/18098), BMP-9 (disclosed in PCT WO 93/00432), BMP-10 (disclosed in PCT WO 94/26893) BMP-11 (disclosed in PCT WO 94/26892), BMP-12 and BMP-13 (disclosed in PCT WO 95/16035), BMP-15 (disclosed in U.S. Pat. No. 5,635,372), BMP-16 (disclosed in U.S. Pat. No. 6,331,612), MP52 (disclosed in PCT WO 93/16099), and BMP-17 and BMP-18 (disclosed in U.S. Pat. No. 6,027,917).

In general, BMP family members initiate their cellular activities by binding to cell surface receptors that possess intrinsic kinase activity in the cytoplasmic domain. The receptors, in turn, initiate signaling events that ultimately lead to changes in gene expression that result in the induction of growth and/or differentiation of the cells.

BMPs are currently in development as protein-based pharmaceuticals. BMP-2 is used clinically for bone repair, and other BMPs are in various stages of clinical development. When protein and DNA are used as pharmaceuticals, a key issue for regulatory agency approval is the ability to produce and standardize batches of the protein or DNA when the batch sizes are increased for large scale manufacturing. One parameter that must be standardized is the biological activity of the pharmaceutical composition.

However, in the case of BMP-based therapeutics, the actual growth inductive activity of BMPs may not be an appropriate assay, primarily because BMP-induced tissue growth occurs slowly over weeks and months. BMP-2 activity may be measured by an alkaline phosphatase-based assay, but other BMPs, including BMP-12 and BMP-13, are not active in this assay. Other methods for measuring BMP activity include cell based assays where addition of BMPs causes a change in an observable phenotype of the cells, in particular, the inhibition of myoblast differentiation of mouse L6 cells. Inada et al., “Bone Morphogenetic Protein-12 and -13 Inhibit Terminal Differentiation of Myoblasts But Do Not Induce Their Differentiation into Osteoblasts” Biochem Biophys Res Comm 222:317-22 (1996). However, these assays are still time-consuming and require subjective analysis of the phenotype, which prevents their use in high throughput or automated screening assays. Accordingly, a need exists for a rapid, simple, quantitative assay for measuring BMP activity in cells.

### SUMMARY OF THE INVENTION

In the experiments leading to the present invention, cells were incubated with exogenous BMPs and the resulting changes in gene expression were evaluated by microarray analysis. The present invention is based, in part, on the discovery that BMPs induce the expression of sFRP2 in this microarray assay.

In additional experiments, cells were incubated with BMPs and the levels of sFRP2 and sFRP3 RNA were evaluated by real-time RT-PCR. Accordingly, the present invention is also based, in part, on the discovery that BMPs induce the expression of sFRP2 and sFRP3 in this RT-PCR assay.

In further experiments, cells were incubated with BMPs and the levels of secreted sFRP2 protein were evaluated by ELISA. Thus, the present invention is based, in part, on the discovery that the BMP-induced increase in sFRP2 RNA levels also result in elevated levels of secreted sFRP2 protein.

Accordingly, the invention provides an assay system for evaluating the biological activity of BMPs by measuring expression of the sFRP2 and/or sFRP3 genes in cells incubated with BMPs.

The invention further provides methods for evaluating the presence of BMP activity in test cells by (1) measuring the levels of sFRP2 and/or sFRP3 gene expression in the test cells; (2) measuring the levels of sFRP2 and/or sFRP3 gene expression in control cells; and (3) comparing the expression levels in the test and control cells, wherein a higher level of sFRP2 and/or sFRP3 expression in the test cells than in the control cells indicates the presence of BMP activity. In some embodiments, the levels of sFRP2 gene expression are detected. In other embodiments, the levels of sFRP3 gene expression are detected. In additional embodiments, the levels of both sFRP2 and sFRP3 are detected. Control cells should not demonstrate any detectable BMP activity in known assays, such as, for example, the alkaline phosphatase assay.

The methods of the invention may be used for detecting the activity of exogenous BMP added to the cells. In some embodiments, the cells are incubated with BMP proteins. In other embodiments, the cells are transfected with DNA encoding the BMP. In one aspect of the invention, the gene expression levels of sFRP2 and/or sFRP3 are compared in the same cells before and after the addition of BMPs to the cells. In other embodiments, the gene expression levels of sFRP2 and/or sFRP3 are compared in test cells incubated with BMP and a separate culture of negative control cells. BMP activity is measured as an increase in sFRP2 and/or sFRP3 expression levels in the cells incubated with BMPS.

In another aspect, the invention provides methods for detecting endogenous BMP activity. In one embodiment, endogenous BMP activity is assessed by measuring the expression levels of sFRP2 and/or sFRP3 in one sample of cells, and comparing those expression levels to the expression levels of sFRP2 and/or sFRP3 in a separate type of cells known to lack the ability to express or respond to BMPs. In particular embodiments, the cells lacking the ability to express or respond to BMPs are COS cells.

In yet another aspect, the invention provides methods for evaluating the efficacy of a test compound to inhibit or stimulate BMP activity in vitro or in vivo comprising:

(a) incubating four sets of cells as follows:
set (1) cells with BMP alone,
set (2) cells with BMP+ test compound,
set (3) cells with test compound alone, and
set (4) cells alone (i.e., without BMP or test compound);

(b) measuring the levels of sFRP2 and/or sFRP3 gene expression in each set of cells; and

(c) if set (3) and set (4) show comparable levels of sFRP2 and/or sFRP3 gene expression, comparing the levels of expression of sFRP2 and/or sFRP3 in set (1) and set (2), wherein a change in the levels of sFRP2 and/or sFRP3 gene expression in set (2) when compared to set (1) indicates that the test compound has BMP modulatory activity.

The cells used in the methods of the invention may be isolated, cultured, or contained within a tissue, organ, and/or patient. In particular embodiments, the methods of the invention can be used in mammalian tissues, particularly bone, cartilage, tendon, or ligament tissue. In other embodiments, the methods of the invention can be used in cultured mammalian cells, particularly murine or human cells.

Expression of sFRP2 and/or sFRP3 gene expression may be measured at RNA or protein levels. In some embodiments, the gene expression is measured at the RNA level. In particular embodiments, the levels of RNA are measured by microarray analysis, real-time RT-PCR, or Northern blot.

In another aspect of the invention, the gene expression is measured at the protein level. In some embodiments, the levels of secreted proteins are measured. In other embodiments, the levels of intracellular or membrane-bound proteins are measured. In yet other embodiments, total protein levels are measured. In particular embodiments, the protein levels are measured by ELISA, immunoblot, immunohistochemistry, immunofluorescence, or mass spectrometry.

The BMP activity measured in the methods of the invention may comprise the activity of at least one of the following BMPs: BMP-2, BMP-4, BMP-5, BMP-6, BMP-7, BMP-8, BMP-9, BMP-10, BMP-11, BMP-12, BMP-13, MP-52, BMP-15, BMP-16, BMP-17, and BMP-18. In particular embodiments, the BMP activity to be measured by the methods of the invention is BMP-2 activity, BMP-12 activity, BMP-13 activity, or MP-52 activity.

The methods of the invention include the use of cells containing native sFRP2 and/or sFRP3 genes as naturally present in the genomic DNA of the cell. In another aspect, the invention includes the use of cells transfected with intact sFRP2 and/or sFRP3 genes. In yet another aspect, the invention includes the use of cells containing reporter constructs comprising sFRP2 and/or sFRP3 promoter sequences linked with reporter genes so that the activation of the sFRP promoter results in the expression of the reporter gene and production of the reporter protein.

The invention may also comprise a kit for detecting the presence of BMP activity. The kit may comprise, in some embodiments, cells responsive to BMPs, primers for detection of sFRP2 and/or sFRP3 expression, and instructions for detecting the RNA levels of sFRP2 and/or sFRP3. In other embodiments, the kit may comprise antibodies specific to sFRP2 and/or sFRP3, and instructions for detecting the protein levels of sFRP2 and/or sFRP3.

Additional aspects of the invention will be set forth in the following description, or may be learned by practice of the invention.

### BRIEF DESCRIPTION OF THE DRAWINGS

FIG. 1 shows the results of a microarray experiment demonstrating that BMP-12 upregulates the expression of sFRP2 RNA in four murine cell lines.

FIG. 2 shows the results of a real-time RT-PCR experiment demonstrating that BMP-2 and BMP-12 upregulate the expression of sFRP-2 cells in clone14 cells.

FIG. 3 shows the results of an ELISA assay demonstrating that BMP-12 and BMP-13 upregulate the levels of sFRP2 protein in the supernatant of clone14 cells.

FIG. 4 shows the results of a real-time RT-PCR assay demonstrating that BMP-12 upregulates the levels of sFRP2 and sFRP3 (FRZB) RNA levels in C2C12 cells.

### BRIEF DESCRIPTION OF THE SEQUENCES

SEQ ID NO:1 is the promoter sequence of mouse sFRP2.

SEQ ID NO:2 is the promoter sequence of human sFRP2.

SEQ ID NO:3 is the promoter sequence of mouse sFRP3.

SEQ ID NO:4 is the promoter sequence of human sFRP3.

### DESCRIPTION OF THE EMBODIMENTS

Reference will now be made in detail to the present embodiments of the invention.

I. Definitions

In order that the present invention may be more readily understood, certain terms are first defined. Additional definitions are set forth throughout the detailed description.

The terms “bone morphogenetic protein” and “BMP” refer to any mammalian gene, RNA, or protein of the BMP family of TGF-β proteins, including but not limited to BMPs-2, 3, 4, 5, 6, 7, 8, 9, 10, 11, 12, 13, 14, 15, 16, 17, and 18, and MP52. In particular, a BMP will have an identifying pattern of seven conserved cysteine residues in the mature, carboxy-terminal portion of the protein, as described in Rosen et al., “Bone Morphogenetic Proteins” Principles of Bone Biology 2:919-928 (2002). BMPs are described, for example, in the following publications: BMP-2, BMP-3, BMP-4, BMP-5, BMP-6, and BMP-7 (disclosed, for example, in U.S. Pat. Nos. 5,108,922; 5,013,649; 5,116,738; 5,106,748; 5,187,076; and 5,141,905), BMP-8 (disclosed in PCT WO 91/18098), BMP-9 (disclosed in PCT WO 93/00432), BMP-10 (disclosed in PCT WO 94/26893) BMP-11 (disclosed in PCT WO 94/26892), BMP-12 and BMP-13 (disclosed in PCT WO 95/16035), BMP-15 (disclosed in U.S. Pat. No. 5,635,372), BMP-16 (disclosed in U.S. Pat. No. 6,331,612), MP52 (disclosed in PCT WO 93/16099), and BMP-17 and BMP-18 (disclosed in U.S. Pat. No. 6,027,917). These terms also refer to variants, allelic variants, fragments of, and mutant BMPs, including but not limited to deletion mutants, insertion mutants, and substitution mutants.

The term “BMP activity” refers to the activation of a signaling cascade by the interaction of a BMP with a cell surface receptor. This activity may result in the induction of growth or differentiation of the cell.

The term “cell” refers to a cell or cells that are isolated, cultured, within a tissue, or within a patient. Any reference to the term “cell” is intended to encompass cells in vivo or in vitro in any form or location, but particularly cells that are isolated, cultured, within a tissue, or within a patient.

The term “patient” refers to any human or animal.

The term “sFRP2” refers to any mammalian gene, RNA, or protein of “secreted Frizzled Related Protein-2,” also known as Secreted Apoptosis Related Protein-1 (SARP-1) and Stromal-Derived Factor-5 (SDF-5), described; for example, in PCT WO 98/35043. This term also refers to fragments and mutants of sFRP2.

The term “sFRP3” refers to any mammalian gene, RNA, or protein of “secreted Frizzled Related Protein-3,” also known as FRZB, described, for example, in Ladner et al., “Cloning and expression of the Wnt antagonists sFRP-2 and Frzb during chick development,” Dev Biol 218:183-198 (2000). This term also refers to fragments and mutants of sFRP3.

The term “test” when referring to cells, tissues, or patients, refers to the cells, tissue, or patient in which the presence of BMP activity is unknown. Test cells, tissue, or patient may be evaluated for the levels of endogenous BMP activity, or they may be treated with exogenous BMP DNA, RNA, or protein, with the BMP activity evaluated either before, after, or during treatment.

The term “test compound” refers to any compound or composition, chemical or biological, whose activity can be evaluated in the methods of the invention.

The terms “undetectable BMP activity” and “no detectable BMP activity” refer to levels of BMP activity that do not produce a phenotypic effect in a known assay, such as a tissue induction assay, the mouse cell differentiation assay, or the induction of sFRP2 or sFRP3 gene expression. It does not mean that there is no BMP activity at all, simply that any BMP activity present is not detectable by well-defined methods, such as, for example, the alkaline phosphatase assay.

II. Assays

sFRP2 RNA levels are increased 2-4 times in cells treated with rhBMP-12 when measured by a microchip assay. A wide range of doses (0.1 nM to 100 nM) of rhBMP-2 and rhBMP-12 will increase sFRP2 RNA levels in a TAQMAN® real-time RT-PCR assay. sFRP2 RNA expression levels may be measured by determining the amount of sFRP2 protein secreted into the medium of cells treated with rhBMP-12 or rhBMP-13, using an Enzyme-Linked Immunosorbent Assay (ELISA) using anti-sFRP2 antibodies. The expression of sFRP2 and/or sFRP3 RNA and protein is also increased in cells treated with rhBMP-12 when measured by real-time RT-PCR and ELISA assays.

Thus, the invention provides a method for evaluating BMP activity in cells by measuring the levels of sFRP2 and/or sFRP3 RNA and protein in those cells. These methods can be used to evaluate the activity of exogenous or endogenous BMPs. In one aspect of the invention, the activity of BMP is evaluated by (1) incubating test cells with BMP protein; (2) measuring the levels of sFRP2 and/or sFRP3 gene expression; and (3) comparing those levels to the gene expression levels in control cells that have not been incubated with BMPs. Alternatively, the activity of BMP may be evaluated by (1) measuring the gene expression levels of sFRP2 and/or sFRP3 in control cells that have not been incubated with BMPs; (2) incubating those same cells with BMPs; and (3) comparing the gene expression levels in the cells before and after incubation with BMPs. In both cases, an increase in sFRP2 and/or sFRP3 gene expression levels in cells incubated with BMPs reflects the presence of BMP activity.

In another aspect, the invention provides methods for assessing the efficacy of a test compound in modulating BMP activity. This can be done by incubating cells with BMP and test compound (set 1) and cells with BMP without test compound (set 2) and determining the levels of sFRP2 and/or sFRP3 expression in those cells. To ensure that any increase or decrease in sFRP2 and/or sFRP3 expression is attributable to the test compound's effect on BMPs, as opposed to a direct effect on sFRP2 and/or sFRP3 expression, the difference between the sFRP2 and/or sFRP3 expression in cell sets 1 and 2 can be compared to the difference in expression levels between cells incubated with the test compound without BMP (set 3), and control cells incubated without BMP or test compound (set 4). A difference in sFRP2 and/or sFRP3 expression between sets 3 and 4 is less than about 50% of the difference in sFRP2 and/or sFRP3 expression between sets 1 and 2 indicates that the test compound has a BMP modulatory effect. In some embodiments, the difference in expression between sets 3 and 4 is less than about 40%, 30%, 20%, or 10% of the difference in expression between sets 1 and 2. An increase in expression of sFRP2 and/or sFRP3 indicates that the test compound stimulates BMP activity. A decrease in expression of sFRP2 and/or sFRP3 indicates that the test compound inhibits BMP activity.

In a related aspect, the invention also provides methods for assessing the ability of a BMP antibody to bind to and inhibit BMP activity. This assay may be used to determine whether an antibody known to bind to BMP affects BMP activity, or whether a mutation in a BMP prevents an antibody from affecting BMP activity. In addition to BMP-specific antibodies, this assay is useful for assessing the ability of anti-BMP receptor protein antibodies to block an interaction between BMPs and receptors. The antibody assays would be performed identically to those of the test compound. This can be done by incubating cells with BMP and an antibody (set 1) and cells with BMP without the antibody (set 2) and determining the levels of sFRP2 and/or sFRP3 expression in those cells. To ensure that any increase or decrease in sFRP2 and/or sFRP3 expression is attributable to the antibody's effect on BMPs, as opposed to a direct effect on sFRP2 and/or sFRP3 expression, the difference between the sFRP2 and/or sFRP3 expression in cell sets 1 and 2 can be compared to the difference in expression levels between cells incubated with the antibody without BMP (set 3), and control cells incubated without BMP or antibody (set 4). A difference in sFRP2 and/or sFRP3 expression between sets 3 and 4 is less than about 50% of the difference in sFRP2 and/or sFRP3 expression between sets 1 and 2 indicates that the antibody has a BMP modulatory effect. In some embodiments, the difference in expression between sets 3 and 4 is less than about 40%, 30%, 20%, or 10% of the difference in expression between sets 1 and 2. An increase in expression of sFRP2 and/or sFRP3 indicates that the antibody stimulates BMP activity. A decrease in expression of sFRP2 and/or sFRP3 indicates that the antibody inhibits BMP activity.

The methods of the invention may also be used to compare and standardize different batches of the same BMP, or to compare BMP activity among batches of different BMPs. Because the expression of sFRP2 and sFRP3 directly correlates with BMP activity, the expression levels of sFRPs and/or sFRP3 may be used as a quantitative measure of BMP activity. Accordingly, one aspect of the invention provides a method to standardize different batches of BMPs by activity level. In this method, multiple cultures of test cells are incubated with different batches and the expression levels of sFRP2 and/or sFRP3 are measured and compared. Once the levels of expression are known, the batches can be concentrated or diluted so that each batch has the same BMP activity/volume ratio.

In particular embodiments, the invention is carried out as follows. Cells known to express a BMP receptor are cultured in an appropriate medium, such as low-serum medium with a serum concentration of 1% or less (v/v). Non-limiting examples of suitable cell lines include those derived from embryonic and mesenchymal stem cells, osteoblasts, tendon cells, bone marrow stromal cells, and epithelial cells. (See, e.g., Nohe et al., “Signal Transduction of Bone Morphogenetic Protein Receptors” Cellular Signalling 16:291-299 (2004).) BMPs are added to the medium of one set of cells (test cells) while a second set remains untreated (control cells). After an appropriate amount of time, the supernatant and the cells may be harvested and sFRP2 and/or sFRP3 gene expression is measured as described below.

The cells can be incubated with BMPs for an amount of time sufficient to induce the expression of the sFRP2 and/or sFRP3 genes and for as long as the cells survive. In some embodiments, the incubation time is at least about 1 hour, 3 hours, 6 hours, 12 hours, 24 hours, 36 hours, 48 hours, or 1 week. In particular embodiments, the incubation time is about 12 hours to about 48 hours.

The levels of sFRP2 and/or sFRP3 gene expression can be determined by any suitable method. Expression, at the RNA or at the protein level, can be determined using routine methods. Expression levels are usually scaled and/or normalized for the total amount of RNA or protein in the sample and/or control cells by using a housekeeping gene like beta-actin, fibronectin, histone, transferring receptor, or GAPDH. Other examples of housekeeping genes would be well known to the skilled artisan.

In one aspect of the invention, gene expression is measured at the RNA level. In particular embodiments, the levels of RNA are measured by microarray analysis, real-time RT-PCR, or Northern blot. These methods are well known in the art, and systems and reagents for performing these analyses are commercially available from a number of companies.

In particular embodiments, involving analysis of RNA expression levels, suitable cells are plated in culture dishes and grown in media supplemented with either a BMP or no protein. After a suitable amount of time, the cells are lysed and RNA is extracted. Real-time RT-PCR is then performed on each sample using any appropriate RT-PCR system and sFRP2 and/or sFRP3 primers and probes. The levels of expression of sFRP2 and/or sFRP3 RNA and a control housekeeping gene RNA are determined using an appropriate gene expression array or other RT-PCR quantitative measurements. In particular, the-cycle threshold method may be used to normalize sFRP2 and/or sFRP3 gene expression to the expression of the housekeeping gene, then to compare sFRP2 and/or sFRP3 RNA levels in BMP treated cells to levels in untreated cells. The increase in sFRP2 and/or sFRP3 RNA expression in the presence of BMPs is then calculated, and the amount of BMP activity correlated to the increase in sFRP2 and/or sFRP3 RNA levels in BMP-treated cells.

In another aspect, gene expression is measured at the protein level. In particular embodiments, the levels of secreted proteins are measured. In other embodiments, the levels of intracellular proteins are measured. In some embodiments, the protein levels are measured by ELISA, immunoblot, immunohistochemistry, immunofluorescence, or mass spectrometry. These methods are well known in the art, and systems and reagents for performing these analyses are commercially available from a number of companies.

In particular embodiments, an ELISA assay may be used to detect sFRP2 and/or sFRP3 protein levels in the supernatant. Suitable cells are plated in culture dishes and grown in media supplemented with either a BMP or no protein. After an appropriate amount of time, the supernatant is removed from the cells. Titer plates are coated with anti-sFRP2 capture antibody and the supernatants from the experimental samples are incubated in the titer plates. After washing, a second anti-sFRP2 detecting antibody is added to the samples. A suitable detecting antibody and substrate are then added to the samples. A standard curve may be constructed using recombinant mouse sFRP2 or other suitable purified sFRP2 and/or sFRP3 protein. These purified sFRP2 and/or sFRP3 proteins are commercially available, or they may be produced by known methods. In particular, the standard curve may be used to compare sFRP2 and/or sFRP3 protein levels in BMP treated cells to levels in untreated cells. The increase in sFRP2 and/or sFRP3 protein levels in the presence of BMPs is then calculated and the amount of BMP activity correlated to the increase in sFRP2 and/or sFRP3 protein levels in BMP-treated cells.

sFRP2 and sFRP3 specific antibodies are well known in the art and are commercially available (e.g., sFRP2 antibody, R&D Systems, Minneapolis, Minn.; sFRP3 antibody, Santa Cruz Biotechnology, Santa Cruz, Calif.). Alternatively, sFRP2 and sFRP3 specific antibodies may be produced by methods known in the art, including those disclosed in “Antibodies: A Laboratory Manual” eds. Harlow et al., Cold Spring Harbor Laboratory, 1988.

In an alternative embodiment, the BMP activity may be evaluated by methods for measuring the expression of reporter proteins whose expression is driven by sFRP2 and/or sFRP3 promoters. In this embodiment, the methods include (1) transfecting both test and control (no BMP treatment) cells with a reporter construct; (2) incubating the test cells with BMPs; and (3) comparing the reporter gene expression levels in the test cells and control cells by any suitable reporter assay. An increase in reporter gene expression in the test cells reflects the presence of BMP activity.

III. Compositions and Methods

A. Cells

The invention comprises methods for detecting BMP activity in cells. Generally, a multitude of cell types are suitable in the methods of the invention. However, to actually have BMP activity within the cells, the cells must be BMP-responsive. Generally, this will entail expression of a functional BMP receptor on the cell surface. The BMP receptor may be endogenous or transgenic. In a non-limiting example, the cells may be selected based on their endogenous expression of a BMP receptor. Suitable cells that express BMP receptors include, but are not limited to, embryonic and mesenchymal stem cells, osteoblasts, tendon cells, bone marrow stromal cells, and epithelial cells.

In an alternative embodiment, cells that do not express BMP receptors may be transfected with DNA encoding a BMP receptor, which would then be expressed on the cell surface. The DNA may be transiently or stably transfected. DNA encoding BMP receptors has been isolated and cloned, and transfection of mammalian cells is a technique well known in the art. (See, e.g., U.S. Pat. No. 6,291,206.) Generally, most available cells lines can be transfected with DNA for the purposes of expressing proteins from that DNA. Examples of commonly used cell lines include COS cells, HeLa cells, CHO cells, and other cell lines. Such cell lines are available commercially.

Suitable cells may be of animal origin, particularly of mammalian origin. In particular embodiments, the cells are of murine or human origin. The cells may be isolated primary cells or cultured cells. In some embodiments, the cells are within an organ tissue, which may be within a patient.

B. Bone Morphogenetic Proteins

The invention further provides methods for detecting BMP activity by incubating cells with BMP protein or by expressing BMP DNA transgenically.

BMPs are a highly homologous family of proteins, and are separated into subgroups based on even higher levels of homology. Some important subgroups include: BMP-2 and BMP-4; BMP-5, BMP-6, and BMP-7; and BMP-12, BMP-13, and MP-52. In particular, BMPs share an identifying pattern of cysteine residues in the carboxy-terminal region of the protein, which is where the BMP activity resides. Accordingly, the methods of the invention may be used to evaluate the following BMPs: BMP-2, BMP-4, BMP-5, BMP-6, BMP-7, BMP-8, BMP-9, BMP-10, BMP-11, BMP-12, BMP-13, MP-52, BMP-15, BMP-16, BMP-17 and BMP-18. In particular embodiments, the BMP activity to be measured by the methods of the invention is BMP-2 activity, BMP-12 activity, BMP-13 activity, or MP-52 activity.

The amount of BMPs used in the methods of the invention can be determined by routine experimentation by methods known to those of skill in the art. In some embodiments, the BMPs are used at a concentration of about 0.1 nM to about 100 nM. In particular embodiments, the BMP concentration in the assays is at least about 0.05 nM, 0.1 nM, 1 nM, 5 nM, 10 nM, 25 nM, 50 nM, 100 nM, 250 nM, 500 nM, or 1000 nM.

The BMPs may be added to the cells as crude, purified, or recombinant proteins. Alternatively, the BMP proteins may be expressed by transiently or stably transfected DNA encoding the BMP of interest. Finally, the BMP may be endogenously expressed by the cell. Methods for producing crude, recombinant, or purified versions of BMP proteins are well known in the art and systems and reagents for producing these proteins for use in the methods of the invention are well known and commercially available from a number of sources. When transfecting cells with DNA encoding BMPs, conventional gene transfer methods may be used to introduce DNA into cells.

C. sFRP2 and sFRP3

The methods of the invention rely on the regulation of expression of the sFRP2 and sFRP3 genes by BMPs. Accordingly, the invention comprises suitable methods for evaluating the expression of sFRP2 and sFRP3.

sFRP2 and sFRP3 are members of the “Frizzled-Related Protein” family. Generally, these proteins are characterized by their activity as WNT antagonists in the WNT signaling pathway. They have also been implicated in the prevention of apoptosis. However, the regulation of sFRP2 and/or sFRP3 gene expression and the biological activity of sFRP2 and sFRP3 proteins is not well characterized and there are no known prototypical functions for these proteins.

In the methods of the invention, the interaction between BMPs and a BMP receptor on the cell surface initiates a signaling cascade that results in the activation of transcription of the sFRP2 and/or sFRP3 genes. Because the control of gene expression is located in the promoter regions of most genes, the invention also includes reporter constructs comprising the sFRP2 and/or sFRP3 promoter sequences linked to reporter genes. Any detectable reporter gene may be suitable for use in the methods of the invention, including, but not limited to, luciferase, Chloramphenicol AcetylTransferase (CAT), Green Fluorescent Protein (GFP), alkaline phosphatase, β-galactosidase, β-glucoronidase, and DsRed. (See, e.g., Spergel et al., “Using reporter genes to label selected neuronal populations in transgenic mice for gene promoter, anatomical, and physiological studies” Prog Neurobiol 63(6):673-686 (2001); Rosochacki et al., “Green fluorescent protein as a molecular marker in microbiology” Acta Microbiol Pol 51(3):205-216 (2002); Barka et al., “Production of cell lines secreting TAT fusion proteins” J. Histochem Cytochem 52(4):469-77 (2004); Liu et al., “Detection of GDNF secretion in glial cell culture and from transformed cell implants in the brains of live animals” Mol Genet Genomics 266(4): 614-623 (2001).) These reporter genes may be transiently or stably expressed in the cells, and may be linear, episomal, or chromosomally integrated.

Accordingly, in one aspect, the invention includes native sFRP2 and sFRP3 genes as naturally present in the genomic DNA of the cell. In another aspect, the invention includes transfected intact sFRP2 and sFRP3 genes. In yet another aspect, the invention includes reporter constructs comprising sFRP2 and/or sFRP3 promoter sequences linked with reporter genes so that the activation of the sFRP promoter results in the expression of the promoter gene and production of the reporter protein. In particular embodiments, the promoter sequences comprises the nucleotides of SEQ ID NO:1, SEQ ID NO:2, SEQ ID NO:3, and/or SEQ ID NO:4.

The promoter sequences of sFRP2 and sFRP3 contain numerous promoter elements, including SMAD3 and SMAD4 regions. Accordingly, promoter sequences comprising the specific promoter elements of the sFRP2 and sFRP3 promoter regions may be used in the methods of the invention.

In some embodiments, a promoter sequence of the invention may comprise one of the following sequences:

nucleotides 1314-1328, 2109-2123, 2824-2838, 6044-6052, 6270-6284, 6783-6806, 7542-7556, 9259-9267, and/or 10599-11349 of SEQ ID NO:1;

nucleotides 1690-1704, 8001-8015, 8194-8208, 9629-9647, 9678-9686, and/or 11652-11896 of SEQ ID NO:2;

nucleotides 65-79, 451-459,1938-1946, 2751-2759, 2834-2842, 3637-3645, and/or 4001-4560 of SEQ ID NO:3; and/or

nucleotides 1-88, 415-423, 621-1610,1851-1882, 2047-2055, 3231-3239, 3250-3254, and/or 3544-4247 of SEQ ID NO:4.

In particular embodiments, a promoter sequence of the invention may comprise a sequences containing one or more of the promoter elements set forth in the following tables:

TABLE 1

Human sFRP2

position on

| | | |
|---|---|---|
| element | SEQ ID NO:2 | sequence |

| | | |
|---|---|---|
| name | (strand) | ((+) strand is shown) |

| | | |
|---|---|---|
| SMAD-3 | 821 (+) | tTTCTGact |
| SMAD-4 | 852 (+) | gagggccAGACTcca (SEQ ID NO: 5) |
| SMAD-3 | 855 (−) | ggcCAGACt |
| SMAD-3 | 1400 (−) | agtCAGATt |
| SMAD-4 | 1690 (−) | agtTGGCTacactac (SEQ ID NO: 6) |
| SMAD-4 | 2080 (−) | ctcTGACTgccctcc (SEQ ID NO: 7) |
| SMAD-3 | 2083 (+) | tGACTGccc |
| SMAD-4 | 2369 (−) | agtTGGCTcaaggat (SEQ ID NO: 8) |
| SMAD-3 | 2829 (+) | tGTCAGtct |
| SMAD-4 | 3389 (+) | gtaaactAGTCAact (SEQ ID NO: 9) |
| SMAD-4 | 3846 (−) | cgtTGCCTcaacttc (SEQ ID NO: 10) |
| SMAD-3 | 4567 (+) | tTTCTGtct |
| SMAD-4 | 5478 (+) | atcattcAGACAcca (SEQ ID NO: 11) |
| SMAD-3 | 7611 (+) | aGACTGgct |
| SMAD-4 | 8001 (+) | gcggcccAGCCAgct (SEQ ID NO: 12) |
| SMAD-4 | 8194 (−) | agaTGGCTgaatccc (SEQ ID NO: 13) |
| SMAD-3 | 8303 (−) | agcCAGGCg |
| SMAD-4 | 8318 (+) | cggttggAGACAccc (SEQ ID NO: 14) |
| SMAD-4 | 8361 (+) | gcccggtAGTCActt (SEQ ID NO: 15) |
| SMAD-4 | 8654 (−) | agcGGACTccctgac (SEQ ID NO: 16) |
| SMAD-3 | 8701 (+) | tGCCTGtcc |
| SMAD-4 | 8949 (+) | tatgggcAGCCCcct (SEQ ID NO: 17) |
| SMAD-4 | 9400 (+) | gtgggaaAGGCAgca (SEQ ID NO: 18) |
| SMAD-4 | 9491 (+) | gcgggccGGGCAaac (SEQ ID NO: 19) |
| SMAD-4 | 9629 (+) | cgggggcCGCCAgcc (SEQ ID NO: 20) |
| SMAD-4 | 9633 (+) | ggccgccAGCCActt (SEQ ID NO: 21) |
| SMAD-3 | 9636 (−) | cgcCAGCCa |
| SMAD-3 | 9678 (−) | agcCAGACc |
| SMAD-3 | 10505 (−) | agtCAGAGa |
| SMAD-4 | 11408 (+) | agcggccAGGCTtct (SEQ ID NO: 22) |
| SMAD-4 | 11477 (+) | ggggcgcAGCCAgaa (SEQ ID NO: 23) |
| SMAD-4 | 11636 (−) | ctcTGGCTgtgcccc (SEQ ID NO: 24) |
| SMAD-4 | 11761 (+) | cgccggcTGCCAgct (SEQ ID NO: 25) |
| SMAD-3 | 11764 (+) | cGGCTGcca |

TABLE 2

Mouse sFRP2

position on

| | | |
|---|---|---|
| element | SEQ ID NO:1 | sequence |

| | | |
|---|---|---|
| name | (strand) | ((+) strand is shown) |

| | | |
|---|---|---|
| SMAD-4 | 16 (+) | acgtgccAGTCAcat (SEQ ID NO: 26) |
| SMAD-4 | 107 (+) | gaaggccAGACTcca (SEQ ID NO: 27) |
| SMAD-3 | 110 (−) | ggcCAGACt |
| SMAD-3 | 228 (+) | gGGCTGcct |
| SMAD-3 | 716 (+) | gGTCAGtct |
| SMAD-3 | 1234 (+) | tCTCTGcct |
| SMAD-4 | 1235 (−) | ctcTGCCTgcccagc (SEQ ID NO: 28) |
| SMAD-3 | 1238 (+) | tGCCTGccc |
| SMAD-4 | 1314 (+) | ggaaagcAGCCAccc (SEQ ID NO: 29) |
| SMAD-3 | 1732 (−) | ggtCAGGCa |
| SMAD-3 | 1757 (−) | aggCAGAAg |
| SMAD-4 | 1793 (−) | agcCGTCTacaccac (SEQ ID NO: 30) |
| SMAD-3 | 1843 (+) | aGACTGtct |
| SMAD-3 | 1884 (+) | tCTCTGtct |
| SMAD-4 | 2109 (+) | atggtgcAGGCAgcc (SEQ ID NO: 31) |
| SMAD-3 | 2116 (−) | aggCAGGCc |
| SMAD-4 | 2824 (+) | gcagagcAGCCAtct (SEQ ID NO: 32) |
| SMAD-4 | 2904 (−) | ataTGTCTccatcct (SEQ ID NO: 33) |
| SMAD-3 | 3147 (+) | aGACTGgct |
| SMAD-3 | 3755 (−) | aggCAGATg |
| SMAD-3 | 3877 (+) | tCTCTGact |
| SMAD-4 | 3927 (−) | agtTGGCCggtcttc (SEQ ID NO: 34) |
| SMAD-3 | 5331 (−) | agcCAGATa |
| SMAD-3 | 5407 (−) | agcCAGCCt |
| SMAD-4 | 5585 (+) | gctgggtAGGCAgca (SEQ ID NO: 35) |
| SMAD-3 | 6044 (−) | tgaCAGACa |
| SMAD-3 | 6068 (−) | agaCAGAGa |
| SMAD-3 | 6076 (−) | agaCAGAGg |
| SMAD-4 | 6270 (+) | gaagtccAGACAaac (SEQ ID NO: 36) |
| SMAD-4 | 6725 (−) | acaTGGCTtactgag (SEQ ID NO: 37) |
| SMAD-3 | 6764 (−) | agaCAGGCt |
| SMAD-4 | 6783 (+) | gggaggtAGCCAtca (SEQ ID NO: 38) |
| SMAD-3 | 6798 (−) | agtCAGACa |
| SMAD-4 | 6818 (−) | agcTGGCTttgtctc (SEQ ID NO: 39) |
| SMAD-4 | 7339 (+) | gtactagAGACAacc (SEQ ID NO: 40) |
| SMAD-4 | 7378 (−) | agaTGGCTgcagcga (SEQ ID NO: 41) |
| SMAD-4 | 7419 (+) | accgggcAGCCTtac (SEQ ID NO: 42) |
| SMAD-4 | 7456 (+) | ctggagaAGACAacc (SEQ ID NO: 43) |
| SMAD-4 | 7542 (+) | caaaggcAGGCAgct (SEQ ID NO: 44) |
| SMAD-3 | 7545 (−) | aggCAGGCa |
| SMAD-4 | 8147 (+) | cctgggcAGCCAaag (SEQ ID NO: 45) |
| SMAD-3 | 8150 (−) | gggCAGCCa |
| SMAD-3 | 8502 (−) | agaCAGAAa |
| SMAD-3 | 8540 (−) | agaCAGATg |
| SMAD-4 | 9252 (+) | gcgagcgAGACAgac (SEQ ID NO: 46) |
| SMAD-3 | 9259 (−) | agaCAGACg |
| SMAD-4 | 9313 (+) | catgggcAGCCCgct (SEQ ID NO: 47) |
| SMAD-3 | 9639 (−) | aggCAGGCc |
| SMAD-4 | 9964 (+) | gaggggaAGTCActa (SEQ ID NO: 48) |
| SMAD-3 | 10009 (+) | aGGCTGact |
| SMAD-4 | 10038 (+) | ggggaagAGACAccc (SEQ ID NO: 49) |
| SMAD-4 | 10544 (−) | gcaTGGCTgcaattc (SEQ ID NO: 50) |
| SMAD-3 | 10608 (−) | agcCAGTCt |
| SMAD-4 | 10788 (+) | gactgcaAGGCAgct (SEQ ID NO: 51) |
| SMAD-4 | 10832 (−) | cgtTGCCTcctcctc (SEQ ID NO: 52) |
| SMAD-4 | 10990 (+) | tcaacgcAGCCAgcc (SEQ ID NO: 53) |
| SMAD-3 | 10997 (−) | agcCAGCCc |
| SMAD-4 | 11104 (−) | ctcTGGCTgggcccc (SEQ ID NO: 54) |

TABLE 3

Human sFRP3

position on

| | | |
|---|---|---|
| element | SEQ ID NO:4 | sequence |

| | | |
|---|---|---|
| name | (strand) | ((+) strand is shown) |

| | | |
|---|---|---|
| SMAD-3 | 34 (−) | gggCAGACt |
| SMAD-4 | 307 (−) | gctTGACTggccatc (SEQ ID NO: 55) |
| SMAD-4 | 412 (+) | ctcagacAGCCAggt (SEQ ID NO: 56) |
| SMAD-3 | 415 (−) | agaCAGCCa |
| SMAD-3 | 920 (−) | agaCAGAGc |
| SMAD-4 | 1135 (+) | gtcagaaGGACAact (SEQ ID NO: 57) |
| SMAD-3 | 2047 (+) | tGACTGtct |
| SMAD-4 | 2178 (−) | aacTGACTccctgtc (SEQ ID NO: 58) |
| SMAD-4 | 2426 (−) | agaTGGCTaaattga (SEQ ID NO: 59) |
| SMAD-4 | 2472 (−) | aggTGACTtcctgtt (SEQ ID NO: 60) |
| SMAD-4 | 3148 (−) | ataTGGCTccaccgt (SEQ ID NO: 61) |
| SMAD-3 | 3231 (+) | gGTCTGact |
| SMAD-3 | 3673 (−) | tgcCAGCCa |
| SMAD-4 | 3857 (+) | ggggaggAGACAccc (SEQ ID NO: 62) |
| SMAD-4 | 3913 (−) | ttaTGTCTtcctcgc (SEQ ID NO: 63) |
| SMAD-3 | 3946 (+) | tATCTGact |
| SMAD-4 | 3947 (−) | atcTGACTgatctgc (SEQ ID NO: 64) |
| SMAD-4 | 4037 (−) | agcAGCCTgggcggc (SEQ ID NO: 65) |
| SMAD-4 | 4057 (−) | cggTGGCTggagctc (SEQ ID NO: 66) |

TABLE 4

Mouse sFRP3

position on

| | | |
|---|---|---|
| element | SEQ ID NO:3 | sequence |

| | | |
|---|---|---|
| name | (strand) | ((+) strand is shown) |

| | | |
|---|---|---|
| SMAD-4 | 118 (+) | GaggttcAGACAatg (SEQ ID NO: 67) |
| SMAD-4 | 444 (+) | gaagagcAGTCAgtc (SEQ ID NO: 68) |
| SMAD-3 | 451 (−) | agtCAGTCa |
| SMAD-3 | 1786 (+) | aGACTGtct |
| SMAD-4 | 1931 (+) | caggagcAGACAgac (SEQ ID NO: 69) |
| SMAD-4 | 1935 (+) | agcagacAGACAggt (SEQ ID NO: 70) |
| SMAD-3 | 1938 (−) | agaCAGACa |
| SMAD-3 | 2073 (−) | aggCAGAAg |
| SMAD-3 | 2083 (−) | aggCAGATc |
| SMAD-3 | 2134 (−) | ggaCAGCCa |
| SMAD-4 | 2627 (−) | aaaTGGCTccctagc (SEQ ID NO: 71) |
| SMAD-3 | 2751 (−) | agtCAGCCa |
| SMAD-4 | 2776 (+) | gccattgAGCCAgcg (SEQ ID NO: 72) |
| SMAD-4 | 2827 (+) | gaagtagAGACAgac (SEQ ID NO: 73) |
| SMAD-3 | 2834 (−) | agaCAGACt |
| SMAD-3 | 2844 (+) | tATCTGcct |
| SMAD-3 | 2915 (+) | cGTCCGgct |
| SMAD-3 | 3058 (−) | gggCAGCCg |
| SMAD-4 | 3634 (−) | tccTGTCTgactttc (SEQ ID NO: 74) |
| SMAD-3 | 3637 (+) | tGTCTGact |
| SMAD-3 | 4231 (+) | tCTCTGgct |
| SMAD-4 | 4436 (+) | ggggtggGGGCAgct (SEQ ID NO: 75) |

D. Test Compounds

The test compound can be preselected or be part of a larger scale screening of compounds. The methods and assays of the invention can be used to screen panels of test compounds or to confirm the inhibitory or stimulatory activity of a known BMP modulator. The test compound may be part of a library of compounds of interest, or it may be part of a library of structurally-related compounds. The structure of the compound may be known or unknown. Test compounds may be predetermined by known functions or structures. For example, a test compound may be chosen because it binds to a BMP or to a BMP receptor. Additionally, a test compound may be selected because of its homology to a known BMP modulator. Alternatively, selection of the test compound can be arbitrary. In non-limiting examples, the test compound may be a peptide, a protein or protein fragment, a small organic molecule, a chemical composition, a nucleic acid, or an antibody. A number of methods for evaluating the appropriateness of a test compound are well known.

IV. Kits

Another aspect of the invention is a kit for evaluating BMP activity. The kit may comprise, in some embodiments, cells responsive to BMPs, primers for detection of sFRP2 and/or sFRP3 expression, and instructions for detecting the RNA levels of sFRP2 and sFRP3. In other embodiments, the kit may comprise antibodies specific to sFRP2 and/or sFRP3, and instructions for detecting the protein levels of sFRP2 and/or sFRP3. In another aspect, the kit may also comprise sFRP promoter constructs for detection of BMP activity via expression of a reporter gene.

The following examples provide illustrative embodiments of the invention which do not in any way limit the invention. One of ordinary skill in the art will recognize the numerous other embodiments are encompassed within the scope of the invention.

The entire contents of all references, patents and published patent applications cited throughout this application are herein incorporated by reference.

### EXAMPLES

### Example 1

### Microarray Analysis

Four different mouse cell lines (myoblastic precursor cells (C2C12 cells, ATCC), pre-adipocyte cells (3T3L1 cells, ATCC), embryonic fibroblasts (C3H10T1/2, ATCC), and immortalized endochondral skeletal progenitor cells derived from mouse limb bud (clone14)) were cultured in Dulbecco's-modified Eagle's medium (DMEM) supplemented with 10% fetal bovine serum (FBS) for two days at 2000 cells/cm². The medium was changed to. DMEM+1% FBS supplemented with either 10 nM rhBMP-12, 100 nM rhBMP-12, or no protein. Cells from each group were lysed 24 hours after the start of the BMP treatment. Total RNA was extracted RNEASY® Micro Kit (QIAGEN®). Nucleic acid concentration was determined with a spectrophotometer.

A. Array Hybridization

Double stranded DNA was synthesized from 5 μg total RNA using the SUPERSCRIPT® System (INVITROGEN®). The cDNA was purified and transcribed in vitro using T7 RNA polymerase. Biotinylated cRNA was generated using biotin labeled UTP and CTP (Perkin Elimer, Boston, Mass.). Fragmented cRNAs were hybridized to a Murine U74Av2 GENECHIP® or to a murine MOE430A GENECHIP® (AFFYMETRIX®, Santa Clara, Calif.) as recommended by the manufacturer. The chips were scanned using a Hewlett Packard GeneArray Scanner and raw data was generated using AFFYMETRIX® MAS 5.0 Software. Hybridization intensities on each array were further normalized to a standard curve created from a set of 11 transcripts spiked in at defined concentrations. This standard curve was used to convert signal values for each qualifier on each array to frequency units expressed as parts per million. The 5′ to 3′ ratio for GAPDH and β-actin ranged from 0.8 to 1.1.

B. Data Analysis

Pair wise comparisons were performed on log 10 transformed signal values for each of the four cell lines (Clone14, 3T3L1, C3H10T1/2, and C2C12) for control cells not incubated with a BMP versus those incubated with BMP-12. The values of the fold change ratio, P-value based on Student's t test, the number of present calls, and the signal value were calculated for each comparison.

For each fold change ratio (fc(x)), FC(x) was assigned a value based on the following rules:

if fc(x) is greater than 2.95, then FC(x)=6;

if fc(x) is greater than 1.95, then FC(x)=6-(3-fc(x));

if fc(x) is greater than 1.50, then FC(x)=5-((2-fc(x))*6).

For each P-value (pv(x)), PV(x) was assigned a value based on the following rules:

if pv(x) is less than 0.01, then PV(x)=4;

if pv(x) is less than 0.05, then PV(x)=3-((pv(x)-0.01)*25);

The present call value (PC(x)) was calculated according to the Affymetrix algorithm (Affymetrix GCOS® software) and assigned a value of 3 if at least 100% of the samples are called P, assigned 2.5 if 50-75% of the samples are called P, and assigned 1 if only 25-49% of the samples were called P.

The signal value (SV(x)) was calculated according to the Affymetrix algorithm and was assigned 3 if the average frequency of any group had a value of 10 or greater.

Penalty points were assigned if the fold change was less than 1.5, the P-value was greater than 0.05, or the frequency values were less than 10 ppm. The final values calculated from these four parameters (CS(x)) ranged from −16 to +16, with qualifiers have a score of 16 considered the most significant changes.

The scores for the sFRP2 gene are set forth in Table 5.

TABLE 5

| Cell Line | 10 nM BMP-12 | 100 nm BMP-12 |
|---|---|---|
| C2C12 | 16 | 16 |
| Clone14 | 16 | 16 |
| 3T3L1 | 16 | 15.9 |
| C3H10T1/2 | 15.6 | 15.3 |

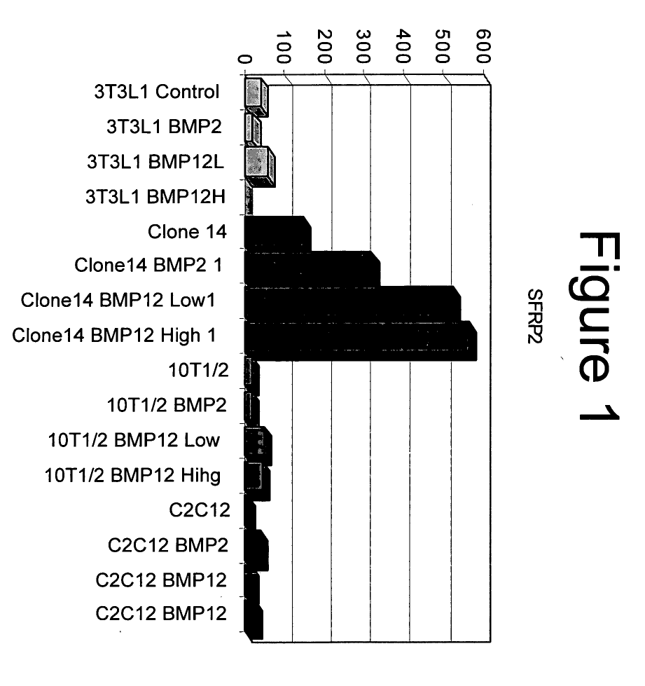

See FIG. 1, which shows that BMP-12 upregulates the expression of sFRP2 RNA in these four murine cell lines.

### Example 2

### Measurement of RNA Levels

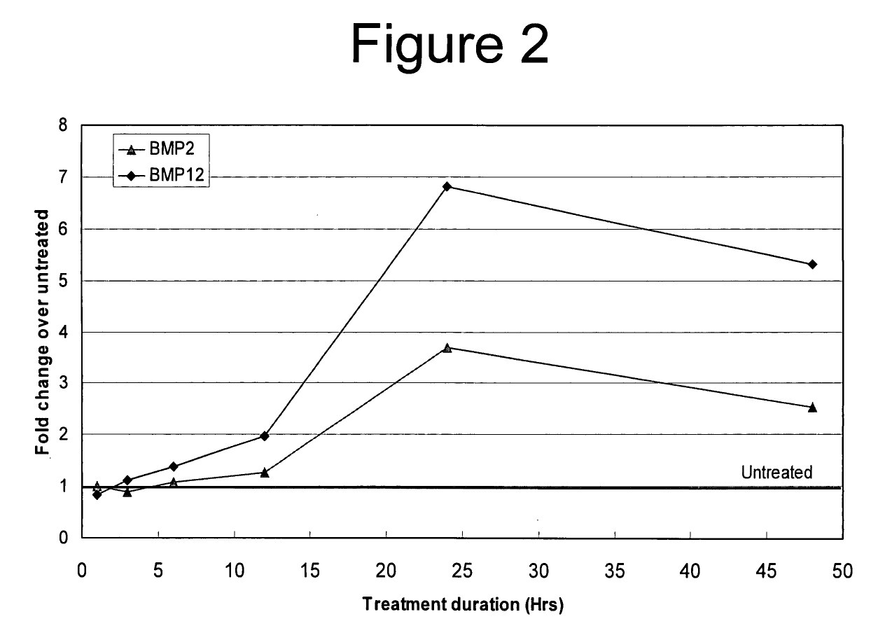

Immortalized endochondral skeletal progenitor cells derived from mouse limb bud (clone14 cells) were plated at 2000 cells/cm2 in 6-well culture dishes. The cells were grown from three days in DMEM+10% FBS. The medium was changed to DMEM+1% FBS supplemented with either 10 nM rhBMP-2, 10 nM rhBMP-12, or no protein. Cells from each group were lysed at 1, 3, 6, 12, 24, and 48 hours after the start of the BMP treatment. Total RNA was extracted and the nucleic acid concentration was determined as described above. Real-time RT-PCR was performed on 100 ng of RNA from each sample in using TAQMAN®. Universal PCR Master Mix. The levels of expression of sFPR-2 RNA and the control gene GAPDH RNA were determined using TAQMAN® Gene Expression Arrays from Applied Biosystems. The cycle threshold method was used to normalize sFRP2 expression to GAPDH, then to compare sFRP2 levels in BMP treated cells to levels in untreated cells. The increase in sFRP2 RNA expression in the presence of BMPs was calculated at 1, 3, 6, 12, 14, and 48 hours. See FIG. 2, which shows that BMP-2 and BMP-12 upregulate the expression of sFRP-2 cells in clone14 cells.

### Example 3

### Measurement of Protein Levels

Clone14 cells were plated at 2000 cells/cm2 in 12-well culture dishes. The cells were grown for four days in DMEM with 10% FBS. The medium was changes to DMEM/Ham's F12 supplemented with 0.1% BSA and either rhBMP-12 or rhBMP-13 at doses of 0, 10, 100, or 1000 nM. After 48 hours, the cell supernatants were collected. The quantity of sFRP2 protein in the supernatant was evaluated using a sandwich ELISA assay.

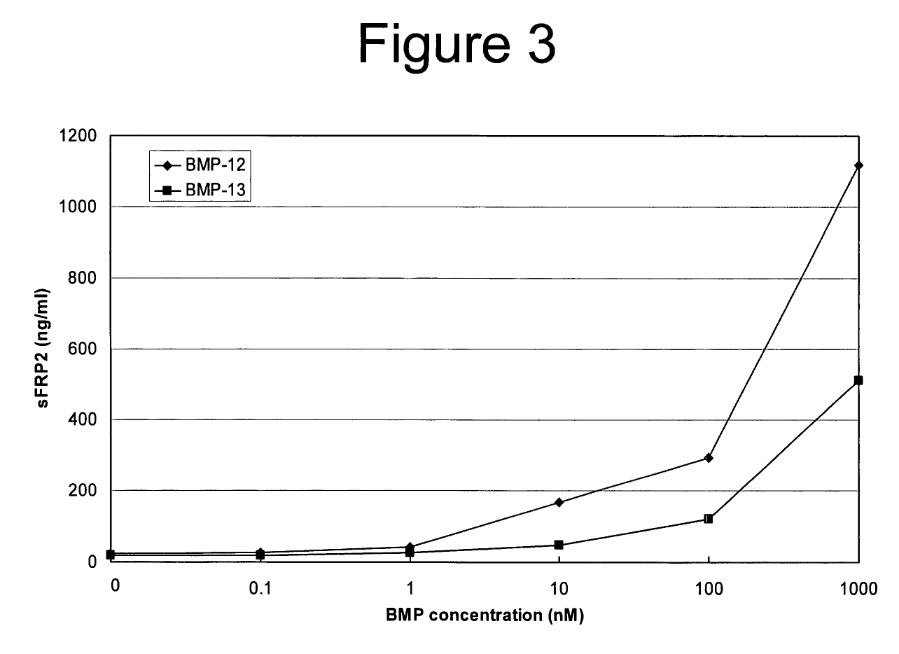

To produce antibodies for the ELISA assay, polyclonal antibodies to human sFRP2 were raised in rabbits and chickens, then affinity-purified using a group of pooled peptides unique to the sFRP2 protein. Titer plates were coated with rabbit anti-sFRP2 capture antibody in phosphate-buffered saline (PBS) overnight at 4° C. After blocking for 1 hour with 2% BSA, the experimental samples were incubated in the titer plates at room temperature for two hours. After washing, a chicken anti-sFRP2 detecting antibody was added to the samples and they were incubated at room temperature for one hour. A horseradish peroxidase (HRP)-conjugated rabbit anti-chicken antibody and tetramethylbenzidene (TMB) substrate were added to the sample for color detection. A standard curve ranging from 7.8-1000 ng/ml was constructed using recombinant mouse sFRP2 (R&D Systems, Minneapolis, Minn.). See FIG. 3, which shows that BMP-12 and BMP-13 upregulate the levels of sFRP2 protein in the supernatant of clone14 cells in this ELISA assay.

### Example 4

### sFRP3 Regulation by BMP-12

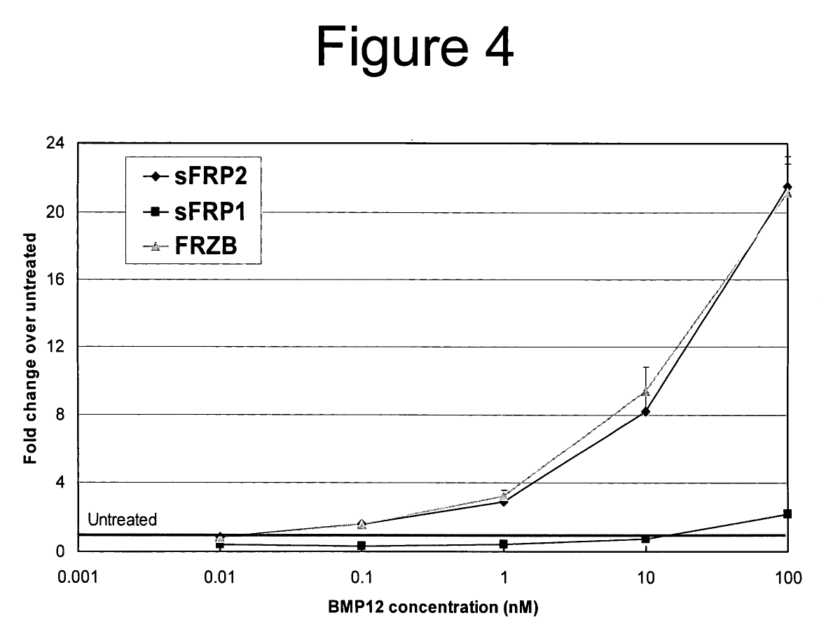

Mouse myoblastic precursor cells (C2C12, ATCC) were cultured in DMEM+10% FBS for two days at 2000 cells/cm2 in 6-well culture dishes. The medium was changed to DMEM+0.1% BSA supplemented with rhBMP-12 at 0, 10, 100, or 1000 nM. After 72 hours of treatment, the cells were lysed and the RNA isolated as described above. Real-time RT-PCR was performed on 100 ng of RNA from each sample. The levels of expression for sFRP2, sFRP3, sFRP-1, and GAPDH RNAs were determined using TAQMAN® Gene Expression Arrays. The cycle threshold method was used to normalize sFRP2, SFRP3, and sFRP-1 expression to GAPDH, then to compare these levels in BMP treated cells to levels in untreated cells. See FIG. 4, which shows t that BMP-12 upregulates the levels of sFRP3 RNA in this experiment.

What is claimed is:

1. A method for evaluating the presence of BMP activity in test cells comprising (1) measuring the levels of sFRP2 and/or sFRP3 gene expression in test cells; and (2) measuring the levels of sFRP2 and/or sFRP3 gene expression in control cells with undetectable BMP activity;
wherein a higher level of sFRP2 and/or sFRP3 expression in the test cells than the control cells indicates the presence of BMP activity in the test cells.

2. The method of claim 1, wherein the levels of sFRP2 and/or sFRP3 gene expression are measured in the control cells and the control cells are then incubated with BMPs to become the test cells.

3. The method of claim 1, wherein the control cells are separate cells with undetectable BMP activity.

4. The method of claim 1, wherein the cells comprise mammalian cells.

5. The method of claim 1, wherein sFRP2 gene expression levels are measured.

6. The method of claim 1, wherein sFRP3 gene expression levels are measured.

7. The method of claim 1, wherein exogenous BMP is added to the test cells before measuring sFRP2 and/or sFRP3 expression levels.

8. The method of claim 7, wherein the cells are incubated with BMP protein.

9. The method of claim 7, wherein the cells are transfected with a DNA encoding BMP protein.

10. The method of claim 1 wherein the BMP comprises at least one protein chosen from BMP-2, BMP-4, BMP-5, BMP-6, BMP-7, BMP-8, BMP-9, BMP-10, BMP-11, BMP-12, BMP-13, MP-52, BMP-15, BMP-16, BMP-17, and BMP-18.

11. The method of claim 1, wherein the gene expression is measured at the RNA level.

12. The method of claim 1, wherein the gene expression is measured at the protein level.

13. A method for evaluating the presence of BMP activity in test cultured cells comprising (1) measuring the amount of sFRP2 and/or sFRP3 protein in the supernatant of the test cultured cells; and (2) measuring the amount of sFRP2 and/or sFRP3 protein in the supernatant of control cells with no detectable BMP activity;
wherein a higher level of sFRP2 and/or sFRP3 protein in the supernatant of the test cultured cells than in the control cells indicates the presence of BMP activity.

14. The method of claim 13, wherein the levels of sFRP2 and/or sFRP3 gene expression are measured in the control cells and the control cells are then incubated with BMPs to become the test cells.

15. The method of claim 13, wherein the control cells are separate cells with no detectable BMP activity.

16. The method of claim 13, wherein the cells comprise mammalian cells.

17. The method of claim 13, wherein sFRP2 protein levels are measured.

18. The method of claim 13, wherein sFRP3 protein levels are measured.

19. The method of claim 13, wherein the BMP comprises at least one protein chosen from BMP-2, BMP-4, BMP-5, BMP-6, BMP-7, BMP-8, BMP-9, BMP-10, BMP-11, BMP-12, BMP-13, MP-52, BMP-15, BMP-16, BMP-17, and BMP-18.

20. The method of claim 13, wherein the protein levels are measured by ELISA.

21. A method for evaluating the presence of BMP activity in test cells that do not express a native BMP receptor comprising (1) transfecting test cells and control cells with a DNA molecule encoding a BMP receptor protein; (2) culturing the transfected cells under suitable conditions to allow expression of the BMP receptor protein; (3) incubating the test cells with a BMP; and (4) measuring the levels of sFRP2 and/or sFRP3 gene expression in the test cells and the control cells;
wherein higher levels of sFRP2 and/or sFRP3 gene expression in test cells than in the control cells indicate the presence of BMP activity in the test cells.

22. The method of claim 21, wherein the levels of sFRP2 and/or sFRP3 gene expression are measured in the control cells and the control cells are then incubated with BMPs to become the test cells.

23. The method of claim 21, wherein the control cells are separate cells with no BMP activity.

24. The method of claim 21, wherein the cells comprise mammalian cells.

25. A method for evaluating the presence of BMP activity in test cells comprising (1) transfecting the test cells and control cells with a DNA molecule comprising a sFRP2 and/or sFRP3 promoter sequence linked to a reporter gene; (2) incubating the test cells with a BMP; and (3) measuring the levels of expression of the reporter gene in the test cells and the control cells;
wherein higher levels of reporter gene expression in the test cells than in the control cells indicate the presence of BMP activity in the test cells.

26. The method of claim 25, wherein the promoter sequence comprises nucleotides 1 to 11349 of SEQ ID NO:1.

27. The method of claim 25, wherein the promoter sequence comprises nucleotides 1 to 11896 of SEQ ID NO:2.

28. The method of claim 25, wherein the promoter sequence comprises nucleotides 1 to 4560 of SEQ ID NO:3.

29. The method of claim 25, wherein the promoter sequence comprises nucleotides 1 to 4247 of SEQ ID NO:4.

30. The method of claim 25, wherein the reporter gene is chosen from luciferase, chloramphenicol acetyltransferase (CAT), Green Fluorescent Protein (GFP), alkaline phosphatase, β-galactosidase, β-glucoronidase, and DsRed.

31. A method for evaluating the presence of endogenous BMP activity in a patient comprising (1) measuring the levels of sFRP2 and/or sFRP3 gene expression in a test tissue of the patient; and (2) measuring the levels of sFRP2 and/or sFRP3 gene expression in a control tissue with undetectable BMP activity;
wherein a higher level of sFRP2 and/or sFRP3 expression in the test tissue than in the control tissue indicates the presence of BMP activity in the test tissue.

32. A method for evaluating the efficacy of a test compound to inhibit or stimulate BMP activity in vitro or in vivo comprising comparing levels of expression of sFRP2 and/or sFRP3 in:
(a) cells incubated with a test compound and BMP; and
(b) cells incubated with BMP and without test compund,
wherein a change in the expression of sFRP2 and/or sFRP3 indicates that the compound is effective for modulating BMP activity.

33. The method of claim 32, wherein the BMP modulatory activity is inhibitory.

34. The method of claim 32, wherein the BMP modulatory activity is stimulatory.

35. An isolated DNA molecule comprising of an sFRP2 or sFRP3 promoter linked in correct reading frame to a reporter gene.

36. The DNA molecule of claim 35, wherein the promoter consists essentially of of nucleotides 1 to 11349 of SEQ ID NO:1.

37. The DNA molecule of claim 35, wherein the promoter consists essentially of of nucleotides 1 to 11896 of SEQ ID NO:2.

38. The DNA molecule of claim 35, wherein the promoter consists essentially of of nucleotides 1 to 4560 of SEQ ID NO:3.

39. The DNA molecule of claim 35, wherein the promoter consists essentially of of nucleotides 1 to 4247 of SEQ ID NO:4.

40. The DNA molecule of claim 35, wherein the reporter gene is chosen fromluciferase, chloramphenicol acetyltransferase (CAT), Green Fluorescent Protein (GFP), alkaline phosphatase, β-galactosidase, β-glucoronidase, and DsRed.
